(19) United States
(12) Patent Application Publication
(10) Pub. No.: US 2010/0092497 A1
(43) Pub. Date: Apr. 15, 2010

(54) METHODS OF IMMUNE OR HAEMATOLOGICAL ENHANCEMENT, INHIBITING TUMOUR FORMATION OR GROWTH, AND TREATING OR PREVENTING CANCER

(76) Inventors: Jagat Rakesh Kanwar, Geelong (AU); Geoffrey Wayne Krissansen, Palmerston North (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 12/520,521

(22) PCT Filed: Dec. 21, 2007
(86) PCT No.: PCT/NZ07/00389

§ 371 Date: Dec. 11, 2009

(30) Foreign Application Priority Data
Dec. 22, 2006 (NZ) 552316

(51) Int. Cl. A61K 38/40; A61K 39/00

(52) U.S. Cl. 424/184.1; 514/12

(57) ABSTRACT

Use of lactoferrin or metal ion lactoferrin, preferably iron lactoferrin, preferably bovine lactoferrin, preferably iron bovine lactoferrin, or a metal ion functional variant or functional fragment thereof and at least one anti-tumour food factor selected from soy protein and vitamin D inhibits tumour formation or growth, maintains or improves one or both of the white blood cell count and red blood cell count, stimulates the immune system, and/or treats or prevents cancer. Dietary (foods or food supplements), nutraceutical or pharmaceutical compositions may be used.

### FIELD OF THE INVENTION

The present invention relates to methods of immune or haematological enhancement, inhibiting tumour formation or growth, and treating or preventing cancer by administration of lactoferrin and an anti-tumour food factor, preferably selected from vitamin D or soy protein or a mixture thereof. Use of lactoferrin or metal ion lactoferrin, preferably iron lactoferrin, preferably bovine lactoferrin, preferably iron bovine lactoferrin, or a metal ion functional variant or functional fragment thereof and at least one anti-tumour food factor selected from soy protein and vitamin D inhibits tumour formation or growth, maintains or improves one or both of the white blood cell count and red blood cell count, stimulates the immune system, and/or treats or prevents cancer. The methods and medicinal uses of the invention may be carried out by employing dietary (as foods or food supplements), nutraceutical or pharmaceutical compositions. Compositions useful in the methods of the invention are also provided.

### BACKGROUND OF THE INVENTION

Bovine lactoferrin (bLf) is a single-chain iron-binding glycoprotein of 78 kDa which is present in bovine milk. It is a natural defense protein present in most secretions commonly exposed to normal flora including milk, colostrum, tears, nasal secretions, saliva, bile, pancreatic juice, intestinal mucus, and genital secretions. It is secreted by neutrophils and present at high levels at sites of bacterial infection. It is a multifunctional protein that may regulate iron absorption in the intestine, promote intestinal cell growth, protect against microbial infection, regulate myelopoiesis, regulate systemic immune responses, and can prevent the development of cancer (reviewed in Ward, et al., 2002; Brock, J H, 2002; Weinburg, E D, 2001; Conneely, O M, 2001; Tomita, et al., 2002 and Tsuda, et al., 2002).

It has previously been reported that tumours do not respond well to chemotherapy in all cases. For example, chemotherapy efficacy varies for cancer sufferers depending on the cancer type, the nature and doses of the drugs used for treatment, the mechanisms by which the drugs work, and the therapeutic regimes.

It is known in the field that cancers differ in their sensitivity to chemotherapy, from the usually and often sensitive (e.g. lymphomas, acute lymphoblastic leukemia (ALL), chronic lymphocytic leukemia (CLL), Hodgkin's disease, intermediate and high grade non-Hodgkin's lymphoma, for example, diffuse large cell lymphoma, Burkitt's lymphoma, lymphoblastic lymphoma, choriocarcinoma, embryonal tumours, myelomatosis, oat cell carcinoma of bronchus, testicular carcinoma, Ewing's sarcoma, Wilms' tumor, skin cancer) where complete clinical cures can be achieved to the largely resistant (bladder cancer, esophageal cancer, non-small cell lung cancer, hepatocellular carcinoma, renal carcinoma, pancreatic carcinoma, head and neck cancer, cervical carcinoma, liver carcinoma, lung carcinomas that are not oat cell). It has previously been reported that EL-4 tumours larger than 0.3 cm in diameter become completely non-responsive to immunotherapy and anti-angiogenic therapy (Kanwar, et al., 1999 and Sun, et al., 2001).

Published international patent application WO 03/099323 reported that bovine lactoferrin was inferior to recombinant human lactoferrin in that it caused a lesser increase in the intestinal IL-18 levels and did not increase the serum levels of IL-18. It reported that bovine lactoferrin does not have the same biological activity or effect as human lactoferrin. Published international patent application WO 2006/054908 reported use of iron-saturated lactoferrin in methods of immune or haematological enhancement, inhibition of tumour formation or growth, and to treat or prevent cancer.

It would therefore be desirable to provide an improved method of inhibiting tumour formation or growth using lactoferrin and an anti-tumour food factor or to at least provide the public with a useful choice.

### SUMMARY OF THE INVENTION

Accordingly, one aspect of the invention relates to a method of inhibiting tumour formation, inhibiting tumour growth or treating or preventing cancer in a subject comprising separate, simultaneous or sequential administration to the subject of lactoferrin and at least one anti-tumour food factor, preferably selected from vitamin D or soy protein or a mixture thereof.

The lactoferrin used may comprise one or more lactoferrin polypeptides, one or more functional lactoferrin variants, one or more functional lactoferrin fragments, one or more metal ion lactoferrin polypeptides, one or more functional metal ion lactoferrin variants, or one or more functional metal ion lactoferrin fragments, or any combination of any two or more thereof. The vitamin D used may comprise vitamin D or one or more vitamin D analogues or any combination of any two or more thereof. The soy protein used may comprise soy, soy protein, soy protein concentrate, soy protein isolate, soy milk, soy yoghurt, soy cheese, soy nuts, soybeans, soybean meal, soybean flour, or soy butter, or any combination of any two or more thereof.

Another aspect of the invention relates to a method of stimulating the immune system of a subject comprising separate, simultaneous or sequential administration to the subject of lactoferrin and at least one anti-tumour food factor, preferably selected from vitamin D or soy protein or a mixture thereof. Another aspect of the invention relates to a method of increasing the production of Th1 and Th2 cytokines within a tumor of a subject in need thereof. Another aspect of the invention relates to a method of increasing the production of Th1 and Th2 cytokines within the intestine of a subject. Another aspect of the invention relates to a method of increasing the level of Th1 and Th2 cytokines in the systemic circulation of a subject.

Another aspect of the invention relates to a method of increasing an anti-tumour immune response in a subject comprising separate, simultaneous or sequential administration to the subject of lactoferrin and at least one anti-tumour food factor, preferably selected from vitamin D or soy protein or a mixture thereof. Another aspect of the invention relates to a method of inducing apoptosis in a subject in need thereof. Another aspect of the invention relates to a method of inducing apoptosis of tumour cells in a subject in need thereof. Another aspect of the invention relates to a method of inhibiting angiogenesis in a subject in need thereof. Another aspect of the invention relates to a method of inhibiting tumour angiogenesis in a subject in need thereof.

Another aspect of the invention relates to a method of maintaining or improving one or both of the white blood cell count and red blood cell count of a subject comprising separate, simultaneous or sequential administration to the subject of lactoferrin and at least one anti-tumour food factor, preferably selected from vitamin D or soy protein or a mixture thereof.

Another aspect of the invention relates to a method of increasing the responsiveness of a subject to a cancer therapy comprising separate, simultaneous or sequential administration to the subject of lactoferrin and at least one anti-tumour food factor, preferably selected from vitamin D or soy protein or a mixture thereof, separately, simultaneously or sequentially with administration of the therapy. Another aspect of the invention relates to a method of increasing the sensitivity of a tumour in a subject to a cancer therapy comprising separate, simultaneous or sequential administration to the subject of lactoferrin and at least one anti-tumour food factor, preferably selected from vitamin D or soy protein or a mixture thereof, separately, simultaneously or sequentially with administration of the therapy. Another aspect of the invention relates to a method of speeding the recovery of a subject undergoing cancer therapy comprising separate, simultaneous or sequential administration to the subject of lactoferrin and at least one anti-tumour food factor, preferably selected from vitamin D or soy protein or a mixture thereof, separately, simultaneously or sequentially with administration of the therapy.

Another aspect of the invention relates to use of lactoferrin in the manufacture of a composition for a purpose as herein described, wherein the composition is administered separately, simultaneously or sequentially with at least one anti-tumour food factor. Another aspect of the invention relates to use of lactoferrin and at least one anti-tumour food factor in the manufacture of a composition for a purpose as herein described. Another aspect of the invention relates to use of lactoferrin and at least one anti-tumour food factor in the manufacture of a composition for a purpose as herein described, wherein the composition is formulated to provide separate, simultaneous or sequential administration of the lactoferrin and the anti-tumour food factor. Another aspect of the invention relates to use of lactoferrin and at least one anti-tumour food factor in the manufacture of a composition for a purpose as herein described, wherein the lactoferrin or functional variant or functional fragment is administered separately, simultaneously or sequentially with the anti-tumour food factor. Another aspect of the invention relates to use of lactoferrin and at least one anti-tumour food factor in the manufacture of a composition for a purpose as herein described, wherein the lactoferrin or functional variant or functional fragment thereof is formulated for administration separately, simultaneously or sequentially with the anti-tumour food factor. Another aspect of the invention relates to a composition comprising, consisting essentially of or consisting of lactoferrin and one or more, two or more or three or more anti-tumour food factors. Another aspect of the invention relates to a product comprising, consisting essentially of or consisting of lactoferrin and one or more, two or more or three or more anti-tumour food factors as a combined preparation for simultaneous, separate or sequential use for a purpose as described herein.

The following embodiments may relate to any of the above aspects.

In one embodiment the lactoferrin is selected from the group comprising a lactoferrin polypeptide, a functional lactoferrin variant, a functional lactoferrin fragment, metal ion lactoferrin, a metal ion lactoferrin functional variant, and a metal ion lactoferrin functional fragment, or a mixture thereof. In one embodiment the lactoferrin is apo-lactoferrin. In another embodiment the lactoferrin is naturally iron-saturated. In another embodiment the lactoferrin is substantially fully iron saturated.

In one embodiment the anti-tumour food factor is selected from vitamin D (including vitamin D1 [lumisterol], vitamin D2 [calciferol or ergocalciferol], vitamin D3 [cholecalciferol], vitamin D4 [22-dihydroerogocalciferol] and vitamin D5 [sitocalciferol] and vitamin D5 [7-dehydrositosterol]), vitamin D analogues (including but not limited to those referenced below), soy protein, one or more soybean components (including those selected from the group comprising but not limited to omega-3 fatty acids from soy, isoflavones from soy (e.g. genistein and/or daidzein), and lunasin peptides (such as those described in U.S. Pat. No. 6,107,287 and U.S. Pat. No. 6,544,956 that are incorporated herein by reference, and those having accession numbers AAE49016, AAE49017, AAP62458 and AAP62459)), polyphenols (from green or black tea for example), lycopene (or tomato juice for example), wheat bran, flavonoids (or apple juice for example), inositol, resveratrol (or grape juice for example), propolis, mushroom extract, anthocyanins (or berry juice for example), almonds, ginseng, casein hydrolysate, and combinations thereof.

In one embodiment the anti-tumour food factor is selected from the group comprising anti-tumour foods and anti-tumour food components. Preferably one or more, two or more or three or more anti-tumour food factors are administered. In one embodiment the anti-tumour food may be a functional food or derivative, thereof that has anti-cancerous properties including fruits, vegetables, legumes, nuts, seeds, grains, spices, herbs, fungi, probiotics, apples, apricots, beans (e.g. green bean, black bean), chick peas, berries (e.g. blueberries, raspberries), cruciferous vegetables (e.g. broccoli, brussel sprouts, cabbage, cauliflower, collards, kale, kohlrabi, bok choy, radish, mustards, and turnips), carrot, cheese, corn products, cranberries, egg plant, flaxseed, allium vegetables [e.g. garlic, onion, spring onion (scallions), chive, leek, shallot], ginger (including ginger components gingerol, paradol, and beta-elemene), ginseng, grapefruit, grapes, grape juice, green or black tea, horseradish, kiwifruit, kumara, leeks, lemons, limes, noni fruit, onions, oranges, peanuts, peppers, rye products, salmon, soy milk products, soy nuts, soybeans, squash, tangerines, tomatoes, wheat bran products, rice, papaya, pawpaw, peaches, persimmons, strawberries, taro leaves, green banana, mango, watercress, yams, almonds, and combinations thereof.

In one embodiment the anti-tumour food component may be selected from the group comprising soy protein, one or more soybean components (including those selected from the group comprising but not limited to omega-3 fatty acids from soy, isoflavones from soy (e.g. genistein and/or daidzein), and lunasin peptides (such as those described in U.S. Pat. No. 6,107,287 and U.S. Pat. No. 6,544,956 that are incorporated herein by reference, and those having accession numbers AAE49016, AAE49017, AAP62458 and AAP62459), shark cartilage, garlic extracts, selenium supplementation, tea extracts (e.g. green or black tea, polyphenols, catechins, epigallocatechin gellate), curcuminoids, caffeine, carnosic acid, capsaicin, sesquiterpene lactones (e.g. parthenolide, costunolide, yomogin), cotylenin A, humulone, arginine, glutamine, retinoids from green leaf vegetables, cocoa powder, lycopene, glucosinolates from cruciferous vegetables, organosulphur compounds (allicin, diallyl sulfide, diallyl disulfide, allyl mercaptan), N-acetyl cysteine, allium compounds, carotenoids (including but not limited to beta-carotene), coumarins, dietary fibres, dithiolthiones, flavonoids (e.g. myricetin, quercetin, rutin), indoles, inositol, inositol hexaphosphate, isoflavones (genistein, daidzein), isothiocyanates, monoterpenes (e.g. limonene, perillic acid, methol, carveol), wheat bran, diterpene esters, polyphenols, riboflavin 5′ phosphate, cinnamaldehyde, vanillin, umbelliferone, phenols (e.g. cinnamic acid), polyphenols, plant sterols (e.g. sitostanol, stigmasterol, campesterol), acylglycosylsterols, phytosteroids, protease inhibitors, saponins, isoprenoids, terprenoids, tocotrienols, retinoids, ellagic acid, polyamines, resveratrol, hydroxycinnamic acids [e.g. (E)-ferulic acid and (E)-p-coumaric acid], chlorophyllin, propolis and some of its components (e.g. caffeic acid, phenyl esters, artellipin C), red wine, tannic acid, mushroom extracts, anthocyanins (e.g. cyanidins), mushroom beta-glucans (e.g. lentinan), spinach leaf extracts, natural antioxidant mixture from spinach leaf, noni juice, vitamins A, B6, C, and E, extract of Siamese cassia, extract of Beta vulgaris, extracts of lemon grass and bamboo grass, carnosic acid, capsaicin, sesquiterpene lactones (e.g. parthenolide, costunolide, yomogin), cotylenin A, humulone, omega-3 fatty acids (including eicosapentaenoic acid (EPA) and docasahexaenoic acid (DHA)), and combinations thereof.

In one embodiment the anti-tumour food component is selected from the group comprising vitamin D, vitamin B6, taurine, arginine, glutamine, colostrum whey, full or partial casein hydrolysates, casein peptide(s) known to be immunostimulatory (e.g. immunocasokinins, caseinophosphopeptides, casomorphins, casokinins), colostrinin peptide, colostrum, beta-carotene, calcium and calcium phosphate, folate, cysteine-rich milk proteins, lactoperoxidase, HAMLET (alpha-lactalbumin-oleic acid complex), fragments of plasminogen, prosaposin, saposins, catalase, lactoperoxidase, fatty acid binding protein, ribonuclease, beta-glucuronidase inhibitor, BRCA1, BRCA2, CD36, interferon, tumour necrosis factor, interleukin 2 (IL-2), kininogen and fragments, kininostatin, cystatin, fetuin, neutrophil defensins, interleukin 12 (IL-12), chitinase-like proteins, dystroglycan, prostasin, SPARC-like proteins, and thrombospondin, and combinations thereof.

In one embodiment a method of the invention comprises administration of a composition consisting essentially of or consisting of lactoferrin, a functional lactoferrin variant, a functional lactoferrin fragment, metal ion lactoferrin, a metal ion lactoferrin functional variant, a metal ion lactoferrin functional fragment, or a mixture thereof, and at least one anti-tumour food factor. Preferably the composition consists essentially of or consists of one or more, two or more or three or more anti-tumour food factors.

In one embodiment of a use of the invention, a composition is manufactured for inhibiting tumour formation in a subject, inhibiting tumour growth in a subject, treating or preventing cancer in a subject, stimulating the immune system in a subject, increasing the production of Th1 and Th2 cytokines within a tumor in a subject, increasing the production of Th1 and Th2 cytokines within the intestine of a subject, increasing the level of Th1 and Th2 cytokines in the systemic circulation of a subject, increasing an anti-tumour immune response in a subject, inducing apoptosis in a subject, inducing apoptosis of tumour cells in a subject, inhibiting angiogenesis in a subject, inhibiting tumour angiogenesis in a subject, maintaining or improving one or both of the white blood cell count and red blood cell count of a subject, increasing the responsiveness of a subject to a cancer therapy, increasing the responsiveness of a tumour in a subject to a cancer therapy or speeding the recovery of a subject undergoing cancer therapy.

In one embodiment the subject is suffering from or is susceptible to cancer; has undergone therapy, but is in relapse or is susceptible to relapse; has a tumour refractory to therapy with a chemotherapeutic, radiotherapeutic, anti-angiogenic or immunotherapeutic agent; or has previously undergone surgery, unsuccessful surgery or unsuccessful therapy with a chemotherapeutic, radiotherapeutic, anti-angiogenic or immunotherapeutic agent.

In one embodiment the metal ion is an ion selected from the group comprising aluminium, copper, chromium, cobalt, gold, iron, manganese, platinum, ruthenium, and zinc ions, or any combination of any two or more thereof. Preferably the metal ion is an iron ion.

In one embodiment the lactoferrin is any mammalian lactoferrin including but not limited to sheep, goat, pig, mouse, water buffalo, camel, yak, horse, donkey, llama, bovine or human lactoferrin. Preferably the lactoferrin is bovine lactoferrin.

In one embodiment the lactoferrin is apo-lactoferrin. In one embodiment the functional lactoferrin variant or functional lactoferrin fragment is free of metal ions. In one embodiment the lactoferrin or functional variant or functional fragment thereof is at least about 5, 10, or 20% metal ion saturated on a stoichiometric basis. In one embodiment the metal ion lactoferrin or a metal ion functional variant or functional fragment thereof is at least about 25, 30, 35, 40, 45, 50, 55, 60, 65, 70, 75, 80, 85, 90, 91, 92, 93, 94, 95, 96, 97, 98, 99, 99.5 or 100% metal ion saturated on a stoichiometric basis. In one embodiment the metal ion lactoferrin or a metal ion functional variant or functional fragment thereof is at least about 105, 110, 115, 120, 125, 130, 135, 140, 145, 150, 155, 160, 165, 170, 175, 180, 185, 190, 195 or 200% metal ion saturated on a stoichiometric basis.

In one embodiment the method comprises administration of a mixture of metal ion lactoferrin and at least one metal ion functional variant or functional fragment thereof.

In one embodiment the lactoferrin and the anti-tumour food factor provide a synergistic therapeutic effect that is greater than the additive effects of either one alone. For example, there is a greater effect on inhibition of tumour formation or growth, tumour regression, cytolytic effects, immune enhancement, generation of Th1 and Th2 cytokines, or the responsiveness of a subject or a tumour to the treatment method. In one embodiment, the lactoferrin and the anti-tumour food factor allow the administration of a co-administered or sequentially administered cancer therapy to be reduced or increased in dose or in length of administration, as appropriate.

In one embodiment the cancer therapy is an anti-tumour agent or anti-tumour therapy. In one embodiment the lactoferrin, at least one anti-tumour food factor and at least one anti-tumour agent or anti-tumour therapy are administered separately, simultaneously or sequentially. In one embodiment the anti-tumour therapy is selected from therapies such as, but not limited to, surgery, chemotherapies, radiation therapies, hormonal therapies, biological therapies/immunotherapies, cellular therapies, anti-angiogenic therapies, cytotoxic therapies, vaccines, nucleic acid-based vaccines (e.g. nucleic acids expressing a cancer antigen such as DNA vaccines including p185 vaccines), viral-based therapies (e.g. adeno-associated virus, lentivirus), gene therapies, small molecule inhibitor therapies, nucleotide-based therapies (e.g. RNAi, antisense, ribozymes etc), antibody-based therapies, oxygen and ozone treatments, embolization, and/or chemoembolization therapies. In one embodiment the anti-tumour agent comprises one or more angiogenesis inhibitors.

In one embodiment the anti-tumour agent is a chemotherapeutic agent or an immunotherapeutic agent. In one embodiment the at least one anti-tumour agent is a chemotherapeutic agent. Preferably the chemotherapeutic agent is selected from tubulin disruptors, DNA intercalators, and mixtures thereof. In one embodiment tubulin disruptors include but are not limited to those listed in published international patent application WO 2006/054908 that is incorporated by reference herein. In one embodiment DNA intercalators include but are not limited to those listed in published international patent application WO 2006/054908 that is incorporated by reference herein. In one embodiment the chemotherapeutic agent is paclitaxel, doxorubicin, epirubicin, fluorouracil, cyclophosphamide or methotrexate.

In one embodiment the anti-tumour agent is an immunotherapeutic agent. Preferably the immunotherapeutic agent is an expression plasmid encoding the T cell co-stimulator B7-1, a T cell co-stimulator, or a functionally related molecule, for example a soluble B7-Ig chimera. In one embodiment the anti-tumour agent comprises immune cell therapy. Preferably the therapy is dendritic cell therapy.

In one embodiment the administration is oral, topical or parenteral administration. In one embodiment a method of the invention further comprises separate, simultaneous or sequential administration of at least one cancer therapy. In one embodiment the at least one anti-tumour agent is administered orally or parenterally, preferably by intravenous, intraperitoneal or intratumoural injection.

In one embodiment the lactoferrin and the anti-tumour food factor are administered daily for at least about 1, 2, 3, 4, 5, 6, 7, 8, 9 or 10 weeks before administration of the anti-tumour agent or anti-tumour therapy. In one embodiment the lactoferrin and the anti-tumour food factor are administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months before administration of the anti-tumour agent or the anti-tumour therapy. In one embodiment the lactoferrin and the anti-tumour food factor are administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months after administration of the anti-tumour agent or the anti-tumour therapy has begun.

In one embodiment the lactoferrin and the anti-tumour food factor are administered at least once daily including continuously over a day orally or by parenteral drip or a combination of administrative routes, with or without a cancer therapy.

In one embodiment the tumour or the cancer is a leukemia, lymphoma, multiple myeloma, a hematopoietic tumor of lymphoid lineage, a hematopoietic tumor of myeloid lineage, a colon carcinoma, a breast cancer, a melanoma, a skin cancer or a lung cancer. In one embodiment the tumour or the cancer is a leukemia such as but not limited to, acute leukemia, acute lymphocytic leukemia, acute granulocytic leukemia, acute myelocytic leukemia such as myeloblastic, promyelocytic, myelomonocytic, monocytic, erythroleukemia leukemia and myelodysplastic syndrome, chronic leukemia such as but not limited to, chronic myelocytic leukemia, chronic granulocytic leukemia, chronic lymphocytic leukemia, and hairy cell leukemia. In one embodiment the tumour or the cancer is a lymphoma such as but not limited to Hodgkin's disease and non-Hodgkin's disease. In one embodiment the tumour or the cancer comprises a hematopoietic tumor of myeloid lineage such as but not limited to acute and chronic myelogenous leukemia, smoldering multiple myeloma, nonsecretory myeloma and osteosclerotic myeloma. In one embodiment the tumour or the cancer comprises a hematopoietic tumor of lymphoid lineage, including leukemia, acute and chronic lymphocytic leukemia, acute and chronic lymphoblastic leukemia, B-cell lymphoma, T-cell lymphoma, Burkitts lymphoma. In one embodiment the tumour or the cancer comprises a hematopoietic tumor of B lymphoid lineage. In one embodiment the tumour or the cancer comprises a hematopoietic tumor of T lymphoid lineage.

In one embodiment the tumour is a large tumour. In one embodiment the tumour is or the cancer comprises (a) a tumour that is at least about 0.3, 0.4 or 0.5 cm in diameter, or (b) a tumour that is refractory to therapy with one at least one immunotherapeutic, anti-angiogenic or chemotherapeutic agent.

In one embodiment one or both of the white blood cell count and red blood cell count of the subject is maintained or improved. In one embodiment the tumour is reduced in size or substantially eradicated.

In one embodiment the lactoferrin is administered in a dosage form comprising digestible protein, preferably casein or other protein such as other edible proteins. In one embodiment the composition is a food, drink, food additive, drink additive, dietary supplement, nutritional product, medical food, nutraceutical, medicament or pharmaceutical. Preferably the composition is formulated for oral or topical administration. Preferably the composition is formulated for oral or parenteral administration. In one embodiment the composition is a milk protein fraction. In one embodiment the lactoferrin is formulated for coadministration with the anti-tumour food factor. In one embodiment the lactoferrin is formulated for sequential administration with the anti-tumour food factor.

In one embodiment the composition comprises a milk composition selected from fresh or recombined whole milk, recombined or fresh skin milk, reconstituted whole or skim milk powder, skim milk concentrate, skim milk powder, skim milk retentate, concentrated milk, ultrafiltered milk retentate, milk protein concentrate (MPC), milk protein isolate (MPI), calcium depleted milk protein concentrate (MPC), low fat milk, low fat milk protein concentrate (MPC), colostrum, a colostrum fraction, colostrum protein concentrate (CPC), colostrum whey, an immunoglobulin fraction from colostrum, whey, whey protein isolate (WPI), whey protein concentrate (WPC), sweet whey, lactic acid whey, mineral acid whey, or reconstituted whey powder.

In one embodiment a composition of the invention or a composition employed in a method of the invention provides a population of lactoferrin polypeptides or functional variants or fragments thereof wherein at least about 25, 30, 35, 40, 45, 50, 55, 60, 65, 70, 75, 80, 85, 90, 91, 92, 93, 94, 95, 96, 97, 98, 99, 99.5 or 100% of the available metal ion-binding pockets in the population are bound to a metal ion, preferably an iron ion.

In one embodiment a composition of the invention or a composition employed in a method of the invention provides a population of lactoferrin polypeptides or functional variants or fragments thereof wherein about 100% of the available metal ion-binding pockets in the population are bound to a metal ion, preferably an iron ion, and additional metal ions are bound to the lactoferrin molecules in non-specific binding sites so that the lactoferrin is at least about 105, 110, 115, 120, 125, 130, 135, 140, 145, 150, 155, 160, 165, 170, 175, 180, 185, 190, 195 or 200% metal ion saturated on a stoichiometric basis.

In one embodiment the composition comprises, consists essentially of or consists of, or a composition used in a method of the invention provides, at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of one or more lactoferrin polypeptides, one or more functional lactoferrin variants, one or more functional lactoferrin fragments, one or more metal ion lactoferrin polypeptides, one or more functional metal ion lactoferrin variants, or one or more functional metal ion lactoferrin fragments, or any combination of any two or more thereof, and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from 20 about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In one embodiment the composition comprises, consists essentially of or consists of, or a composition used in a method of the invention provides, at least about 0.001, 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of one or more anti-tumour food factors selected from vitamin D, one or more vitamin D analogues, soy, soy protein, soy protein concentrate, soy protein isolate, soy milk, soy yoghurt, soy cheese, soy nuts, soybeans, soybean meal, soybean flour, and soy butter, or any combination of any two or more thereof, and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In one embodiment the composition comprises, consists essentially of or consists of, or a composition used in a method of the invention provides, at least about 0.001, 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of one or more additional anti-tumour food factors.

In one embodiment the composition comprises, consists essentially of or consists of, or a composition used in a method of the invention provides a daily dose of lactoferrin of from about 1 mg/kg/day to about 1.5 g/kg/day. In one embodiment the composition comprises, consists essentially of or consists of, or a composition used in a method of the invention provides a daily dose of vitamin D or a vitamin. D analogue or any mixture of any two or more thereof of from about 100 IU/kg/day to about 2,500 IU/kg/day. In one embodiment the composition comprises, consists essentially of or consists of, or a composition used in a method of the invention provides a daily dose of an additional anti-tumour food factor of from about 1 mg/kg/day to about 1.5 g/kg/day.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated, otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1 is two graphs showing that the anti-tumour activity of bovine lactoferrin (Lf) and sensitization of tumours to chemotherapy depends on the level of Fe-saturation. (A) Mice were fed the control AIN93G diet, and the same diet supplemented with either fully Fe-saturated Lf, 50% Fe-saturated Lf, native Lf, or apoLf. Day 0 refers to the day the mice were placed on their diets. After 2 weeks on the diets, EL-4 cells were injected into the flanks of mice. Paclitaxel (30 mg/Kg) was administered as indicated and tumour size monitored for 77 days, or until tumours reached 1 cm in diameter. Each point represents the mean tumour size with 95% confidence intervals for either 10 mice, or the number of mice indicated. (B) Effects on anti-tumor CTL activity. Splenocytes were harvested from mice in FIG. 1A at day 77 (or day 56 in the case of controls) and tested for their cytolytic activity against EL-4 target cells. The percent cytotoxicity is plotted against various effector-to-target cell ratios (E:T ratios). Each point represents the mean percent cytotoxicity obtained from 5 mice. Error bar represents 95% confidence intervals.

FIG. 2 is two graphs showing the dose-response of ˜100% Fe-saturated Lf. (A) Mice were fed the control diet, and the same diet supplemented with different levels of Fe-saturated Lf ranging from 0, 1, 5, 25, and 100 g per 2.4 Kg of diet. Day 0 refers to the day the mice were placed on their diets. After 2 weeks on the diets, EL-4 cells were injected into the flanks of mice. Paclitaxel (30 mg/Kg) was administered as indicated and tumour size was monitored for 77 days, or until tumours reached 1 cm in diameter. Each point represents the mean tumour size with 95% confidence intervals for either 10 mice, or the number of mice indicated. The numbers of mice from each group which completely rejected the tumour challenge is shown above the x-axis. (B) Effects on anti-tumor CTL activity. Splenocytes were harvested from mice in FIG. 2A at day 77 (or day 56 in the case of controls) and tested for their cytolytic activity against EL-4 target cells. The percent cytotoxicity is plotted against various effector-to-target cell ratios (E:T ratios). Each point represents the mean percent cytotoxicity obtained from 5 mice. Error bar represents 95% confidence intervals.

FIG. 3 is two graphs showing that soy protein augments the ability of ˜100% Fe-saturated Lf to inhibit tumorigenesis and renders tumours susceptible to chemotherapy. (A) Mice were fed the control AIN93G diet, or the same diet supplemented with either Fe-saturated Lf, soy protein, or a combination of Fe-saturated Lf and soy protein. Day 0 refers to the day the mice were placed on their diets. After 2 weeks on the diets, 2×10⁵ EL-4 cells were injected into the flanks of mice. Paclitaxel (30 mg/Kg) was administered as a single dose i.p. when the tumours of mice reached ˜0.5 to 0.6 cm in diameter, as indicated by arrows. Tumour size as measured by two perpendicular diameters (in centimetres) was monitored until day 77, or until tumours reached 1 cm in diameter. Each point represents the mean tumour size with 95% confidence intervals for either 10 mice, or the number of mice indicated. (B) Effects on anti-tumor cytolytic activity. Splenocytes were harvested from mice in FIG. 3A at day 77, or day 56 in the case of mice fed the control diet, and tested for their cytolytic activity against EL-4 target cells. The percent cytotoxicity is plotted against various effector-to-target cell ratios (E:T ratios). Each point represents the mean percent cytotoxicity obtained from 10 mice or the number of mice indicated. Error bar represents 95% confidence intervals.

FIG. 4 is three graphs showing that a combination of high-dose vitamin D3 and ˜100% Fe-saturated Lf completely inhibits tumorigenesis. (A) Levels of 1,25(OH)2D3 in serum. Mice were fed either the control AIN93G diet, the same diet supplemented with high dose cholecalciferol at 45.2 mg/Kg, or the AIN93G diet containing increasing amounts of cholecalciferol supplemented with Fe-saturated Lf, as indicated. Serum was collected at the time of sacrifice, and levels of 1,25(OH)2D3 were measured by enzyme immunoassay. (B) Assessment of inhibition of tumorigenesis. Mice were fed diets as described in (A), where Day 0 refers to the day the mice were placed on their diets. After 2 weeks on the diets, 2×10⁵ EL-4 cells were injected into the flanks of mice. Paclitaxel (30 mg/Kg) was administered as a single dose i.p. when the tumours of mice reached ˜0.4 cm in diameter. Tumour size as measured by two perpendicular diameters (in centimetres) was monitored until day 77, or until tumours reached 1 cm in diameter. Each point represents the mean tumour size with 95% confidence intervals for either 6 mice, or the number of mice indicated. (C) Effects on anti-tumor cytolytic activity. Splenocytes were harvested from mice in FIG. 4A at day 77, or day 56 in the case of controls, or when tumours reached 1 cm in diameter, and tested for their cytolytic activity against EL-4 target cells. The percent cytotoxicity is plotted against various effector-to-target cell ratios (E:T ratios). Each point represents the mean percent cytotoxicity obtained from 6 mice, or the number of mice indicated. Error bar represents 95% confidence intervals.

### DETAILED DESCRIPTION OF THE INVENTION

### 1. Definitions

The terms “anti-tumour food factor”, “anti-tumour food” and “anti-tumour food component” refer to foods and food components that are capable of inhibiting tumour formation or growth and preferably are capable of augmenting the ability of lactoferrin to inhibit tumour formation or growth.

The term “anti-tumour factors” refers at least to apoptosis inducing factors and may include anti-tumour cytolytic antibodies and tumoricidal cytokines such as TNF-alpha.

The term “anti-tumour immune response” refers to the ability of lactoferrin to stimulate the generation of antigen-specific cytolytic activity (the activity of immune cells, particularly cytotoxic T-lymphocytes) and/or NK cell activity, improve the cellular immune response to antigens (through the activity, of at least cytotoxic T-lymphocytes), improve immune protection (by at least restoring the activity of cytotoxic T-lymphocytes and/or NK cells and enhancing cytokine production), restore immune protection (by at least restoring or stimulating the activity of cytotoxic T-lymphocytes and/or NK cell activity and enhancing cytokine production), generate pro-inflammatory and immunoregulatory mediators (Th1 and Th2 cytokines), and/or generate anti-tumour cytolytic antibodies and tumoricidal cytokines such as TNF-alpha.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

An “effective amount” is the amount required to confer therapeutic effect. The interrelationship of dosages for animals and humans (based on milligrams per meter squared of body surface) is described by Freireich, et al. (1966). Body surface area can be approximately determined from height and weight of the subject. See, e.g., Scientific Tables, Geigy Pharmaceuticals, Ardley, N.Y., 1970, 537. Effective doses also vary, as recognized by those skilled in the art, dependent on route of administration, excipient usage, and the like.

The terms “enhance the immune system” and “stimulate the immune system” (and different tenses of these terms) refer to the ability of lactoferrin to stimulate the generation of antigen-specific cytolytic activity (the activity of immune cells, particularly cytotoxic T-lymphocytes) and/or NK cell activity, improve the cellular immune response to antigens (through the activity of at least cytotoxic T-lymphocytes), improve immune protection (by at least restoring the activity of cytotoxic T-lymphocytes and/or NK cells and enhancing cytokine production), restore immune protection (by at least restoring or stimulating the activity of cytotoxic T-lymphocytes and/or NK cell activity and enhancing cytokine production) or generate pro-inflammatory and immunoregulatory mediators (Th1 and Th2 cytokines).

The term “functional lactoferrin fragment” is intended to mean a naturally occurring or non-naturally occurring portion of a lactoferrin polypeptide that has activity when assayed according the examples below, and includes metal ion functional fragments. Useful lactoferrin fragments include truncated lactoferrin polypeptides, metal ion-binding hydrolysates of lactoferrin, fragments that comprise the N-lobe metal ion binding pocket, fragments that comprise the C-lobe metal ion binding pocket, and metal ion-binding fragments generated (by artificial or natural processes) and identified by known techniques as discussed below. Published international patent applications WO 2006/054908 and WO 2007/043900 report preparation and use of lactoferrin fragments and are incorporated herein by reference.

The term “functional lactoferrin variant” is intended to mean a variant of a lactoferrin polypeptide that has activity when assayed according the examples below and so is able to inhibit tumour formation or inhibit tumour growth, and includes metal ion functional variants.

The term “glycosylated” when used in relation to a lactoferrin polypeptide, functional variant or functional fragment is intended to mean that the lactoferrin is fully or partially glycosylated with naturally occurring or non-naturally occurring human or bovine glycosyl groups. Glycosylated and aglycosyl forms of lactoferrin are known (see Pierce, et al. (1991); Metz-Boutigue, et al. (1984); van Veen, et al. (2004)).

The term “increasing the responsiveness of a subject” is intended to mean that a subject exhibits a greater reduction in the rate of tumour growth, in tumour size, or in clinical symptoms of disease than a subject who is not subjected to a method of the invention. In one embodiment, the treated subject also benefits from one or more of restored vitamin status, reduced time on chemotherapy, reduced chemotherapy dose, increased immune stamina, increased nutritional health, reduced cachexia, and increased hemapoesis.

The term “increasing the sensitivity of a tumour” is intended to mean that a tumour exhibits a greater reduction in the rate of tumour growth, in tumour size, or is eradicated whereas a tumour that is not subjected to a method of the invention will not exhibit these effects.

The term “immunotherapeutic agent” is intended to mean an agent that stimulates anti-tumour immunity. Agents that stimulate anti-tumour activity are preferably those that directly or indirectly stimulate T-cells and/or NK cells to kill tumour cells. An in vitro assay for assessing whether a selected agent stimulates anti-tumour immunity is the CTL assay described below.

The term “inhibiting tumour formation” is intended to mean that tumours do not form, or that tumours form but do not establish or grow, or that tumours form but remain small, benign and do not become cancerous or metastasize, or that tumours grow more slowly. Tumour formation may be monitored through CT scans and tumor markers where available.

The term “inhibiting tumour growth” is intended to mean that tumours do not form in a subject treated according to the invention, or that one or more tumours that may be present in a subject treated according to the invention do not grow in size or become cancerous or metastasize, or that one or more tumours present in a subject treated according to the invention reduce in size (preferably by at least about 20, 30, 40, 50, 60, 70, 80, 90 or 100% by volume) or that one or more tumours present in a subject treated according to the invention are eradicated. Tumour size may be monitored through CT scans and tumor markers where available.

The terms “iron-lactoferrin” and “iron-saturated lactoferrin” as used herein are intended to refer to a population of lactoferrin polypeptides providing a population of iron-binding pockets where at least about 25, 30, 35, 40, 45, 50, 55, 60, 65, 70, 75, 80, 85, 90, 91, 92, 93, 94, 95, 96, 97, 98, 99, 99.5, 99.9 or 100% of the metal ion-binding pockets present in the population have an iron ion bound.

The term “lactoferrin polypeptide” refers to a non-glycosylated or glycosylated wild-type lactoferrin amino acid sequence or homologous lactoferrin sequences froth other species such as those described below. A lactoferrin polypeptide has two metal-ion binding pockets and so can bind metal ions in a stoichiometric ratio of 2 metal ions per lactoferrin molecule. One metal ion-binding pocket is present in the N-terminal lobe (N-lobe) of lactoferrin and the other pocket is present in the C-terminal lobe (C-lobe) (Moore et al, 1997). Verified sequences of bovine and human lactotransferrins (lactoferrin precursors), lactoferrins and peptides therein can be found in Swiss-Prot (http://au.expasy.org/cgi-bin/sprot-search-ful). Indicative lactoferrin polypeptides include the bovine lactotransferrin precursor accession number P24627, bovine lactoferrin, the human lactotransferrin precursor accession number P02788 and human lactoferrin. Published international patent applications WO 2006/054908 and WO 2007/043900 report preparation and use of lactoferrin polypeptides and are incorporated herein by reference.

The term “large tumour” is intended to mean a tumour that is refractory to therapy with one at least one immunotherapeutic, anti-angiogenic or chemotherapeutic agent, preferably refractory to therapy with at least one at least one immunotherapeutic or chemotherapeutic agent. In one embodiment a large tumour is a tumour that is at least about 0.3, 0.4, 0.5, 0.6, 0.7 or 0.8 cm in diameter. In one embodiment a large tumour is a tumour that is about 0.3 to about 0.8, about 0.4 to about 0.8, about 0.5 to about 0.8, about 0.6 to about 0.8 or about 0.7 to about 0.8 cm in diameter.

In one embodiment a large tumour is a tumour that is refractory to therapy by immunotherapy or anti-angiogenic therapy or chemotherapy.

The term “metal ion-binding” is intended to refer to binding of a metal ion in an iron binding pocket of a lactoferrin polypeptide or in an iron binding pocket of a fragment of a lactoferrin polypeptide that is still able to form the iron binding pocket.

The terms “metal ion lactoferrin” and “metal ion-saturated lactoferrin” are intended to refer to a population of lactoferrin polypeptides that provide a population of metal ion-binding pockets where at least about 25% of the metal ion-binding pockets present in the population have a metal ion bound. It should be understood that the population may contain polypeptides of different species; for example, some molecules binding no ion and others each binding one or two ions. In cases where different metal ions are used, some molecules may bind a metal ion selected from, for example, the group comprising aluminium, copper, chromium, cobalt, gold, iron, manganese, platinum, ruthenium and zinc ions, and others may bind a different ion.

Equally, the terms “metal ion lactoferrin fragment” and “metal ion-saturated lactoferrin fragment” are intended to refer to a population of lactoferrin polypeptide fragments that provide a population of metal ion-binding pockets where at least about 25% of the metal ion-binding pockets present in the population have a metal ion bound.

The present invention may employ a mixture of lactoferrin polypeptides and lactoferrin fragments. In such an embodiment, the population of metal ion-binding pockets is made up of two pockets for every lactoferrin polypeptide and one or two pockets for every lactoferrin fragment, depending on the nature of the fragments.

The degree of saturation may be determined by spectrophotometric analysis (Brock & Arzabe, 1976; Bates et al, 1967; Bates et al, 1973). It should be understood that there may be metal ion-exchange between lactoferrin polypeptides. In one embodiment, iron saturated lactoferrin may be prepared by the method of Law, et al (1977). In another embodiment, iron saturated lactoferrin may be prepared by the method of Kawakami et al (1993). Metal-ion saturated lactoferrin may be prepared by binding metal ions to the metal ion binding sites in lactoferrin, including the metal ion binding pockets such as the Fe binding pockets and other non-specific binding sites on the lactoferrin molecule or lactoferrin fragment.

In one embodiment at least about 25, 30, 35, 40, 45, 50, 55, 60, 65, 70, 75, 80, 85, 90, 91, 92, 93, 94, 95, 96, 97, 98, 99, 99.5, 99.9 or 100% of the metal ion-binding pockets present in the population of lactoferrin molecules have a metal ion bound and useful ranges may be selected between any of the foregoing values (for example, about 25 to about 100%, about 30 to about 100%, about 35 to about 100%, about 40 to about 100%, about 45 to about 100%, about 50 to about 100%, about 55 to about 100%, about 60 to about 100%, about 65 to about 100%, about 70 to about 100%, about 75 to about 100%, about 80 to about 100%, about 85 to about 100%, about 90 to about 100%, about 95 to about 100% and about 99 to about 100%). In one embodiment the metal ion lactoferrin is super-saturated lactoferrin.

The term “metal ion lactoferrin functional fragment” is intended to mean a naturally occurring or non-naturally occurring portion of a lactoferrin polypeptide that has one or two metal ion binding pockets and that has activity when assayed according the examples below. Useful lactoferrin fragments include truncated lactoferrin polypeptides, metal ion-binding hydrolysates of lactoferrin, fragments that comprise the N-lobe metal ion binding pocket, fragments that comprise the C-lobe metal ion binding pocket, and metal ion-binding fragments generated (by artificial or natural processes) and identified by known techniques as discussed below.

The term “oral administration” includes oral, buccal, enteral and intra-gastric administration.

The term “parenteral administration” includes but is not limited to topical (including administration to any dermal, epidermal or mucosal surface), subcutaneous, intravenous, intraperitoneal, intramuscular and intratumoural (including any direct administration to a tumour) administration.

The term “pharmaceutically acceptable carrier” is intended to refer to a carrier including but not limited to an excipient, diluent or auxiliary that can be administered to a subject as a component of a composition of the invention. Preferred carriers do not reduce the activity of the composition and are not toxic when administered in doses sufficient to deliver an effective amount of a lactoferrin polypeptide or functional variant or functional fragment thereof. The formulations can be administered orally, nasally or parenterally.

The term “subject” is intended to refer to an animal, preferably a mammal, more preferably a mammalian companion animal or human. Preferred companion animals include cats, dogs and horses.

The term “super-saturated lactoferrin” refers to a population of lactoferrin polypeptides or functional fragments providing a population of metal ion-binding pockets where sufficient metal ions are available to fill 100% of the binding pockets and additional metal ions are present and bound by non-specific binding sites on the lactoferrin polypeptide or lactoferrin fragment. In other words, a stoichiometric excess of metal ions is provided. Preferably no free metal ions are present in a composition of the invention comprising super-saturated lactoferrin, although metal ion exchange between binding pockets, between non-specific binding sites and between binding pockets and non-specific binding sites may occur. Preferably super-saturated lactoferrin does not form insoluble aggregates. In one embodiment the super-saturated lactoferrin is at least about 105, 110, 115, 120, 125, 130, 135, 140, 145, 150, 155, 160, 165, 170, 175, 180, 185, 190, 195 or 200% metal ion saturated, preferably iron saturated. Useful saturation ranges include about 25 to about 200%, about 30 to about 200%, about 35 to about 200%, about 40 to about 200%, about 45 to about 200%, about 50 to about 200%, about 55 to about 200%, about 60 to about 200%, about 65 to about 200%, about 70 to about 200%, about 75 to about 200%, about 80 to about 200%, about 85 to about 200%, about 90 to about 200%, about 95 to about 200% and about 100 to about 200% metal ion saturation.

The term “treat” and its derivatives should be interpreted in their broadest possible context. The term should not be taken to imply that a subject is treated until total recovery. Accordingly, “treat” broadly includes amelioration and/or prevention of the onset of the symptoms or severity of a particular condition, or extending a patient's quality of life. The term “treat” also broadly includes the maintenance of good health for sensitive individuals and building stamina for disease prevention. For example, “treating or preventing cancer in a subject” is intended to include inhibiting tumour formation or inhibiting tumour growth or both inhibiting tumour formation and inhibiting tumour growth.

The term “variant” refers to a naturally occurring (an allelic variant, for example) or non-naturally occurring (an artificially generated mutant, for example) lactoferrin polypeptide or lactoferrin fragment that varies from the predominant wild-type amino acid sequence of a lactoferrin polypeptide of a given species (such as those listed below) or fragment thereof by the addition, deletion or substitution of one or more amino acids.

Generally, polypeptide sequence variant, possesses qualitative biological activity in common when assayed according to the examples below. Further, these polypeptide sequence variants may share at least about 50%, 55%, 60%, 65%, 70%, 75%, 80%, 85%, 90%, 95%, 96%, 97%, 98% or 99% sequence identity. Also included within the meaning of the term “variant” are homologues of lactoferrin polypeptides. A homologue is typically a polypeptide from a different species but sharing substantially the same biological function or activity as the corresponding polypeptide disclosed herein.

Preferred variant polypeptides preferably have at least about 70, 75, 80, 85, 90, 95 or 99% identity, preferably at least about 90, 95 or 99% identity to a lactoferrin sequence described herein, including those sequences described in published international patent applications WO 2006/054908 and WO 2007/043900 that are incorporated herein by reference. Variant fragments preferably have at least about 70, 75, 80, 85, 90, 95 or 99% identity, preferably at least about 90, 95 or 99% identity to a fragment described herein, including those sequences described in published international patent applications WO 2006/054908 and WO 2007/043900. Identity can be determined by comparing a candidate amino acid sequence to a sequence described herein, such as a lactoferrin polypeptide or fragment thereof using the BLAST suite of programs (version 2.2.12; 28 Aug. 2005) that is publicly available from NCBI (ftp://ftp.ncbi.nih.gov/blast/).

Conservative substitutions of one or several amino acids of a lactoferrin polypeptide sequence without significantly altering its biological activity are also useful. A skilled artisan will be aware of methods for making phenotypically silent amino acid substitutions (see for example Bowie et al., (1990)).

The term “vitamin D” refers to one or more vitamin D compounds selected from the group comprising vitamin D1 [lumisterol], vitamin D2 [calciferol or ergocalciferol], vitamin D3 [cholecalciferol], vitamin D4 [22-dihydroerogocalciferol] and vitamin D5 [sitocalciferol], and any mixture of any two or more thereof. The term “vitamin D analogue” refers to any compound that will bind and activate a vitamin D receptor (VDR). The VDR is a ligand-activated intracellular receptor that acts as a transcription factor and binds vitamin D response elements (VDREs) in the promoter/enhancer regions of genes including but not limited to genes that exert antiproliferative effects on tumour cells by causing arrest in the G0/G1 phase of the cell cycle, down-regulating growth promoting factors such as IGF-1, up-regulating negative growth regulators such as transforming growth factor beta, causing tumour apoptosis, inhibiting tumour angiogenesis and inhibiting metastasis. Assays for assessing VDR binding are known; for example, immunoassays that measure the expression of genes regulated by vitamin D. Therefore, candidate vitamin D analogues may be readily assessed without undue experimentation for use according to the present invention.

### 2. Lactoferrin Polypeptides

In addition to the useful lactoferrin polypeptides and fragments listed above, examples of lactoferrin amino acid and mRNA sequences that have been reported and are useful in methods of the invention include but are not limited to the amino acid (Accession Number NP_{—}002334) and mRNA (Accession Number NM_{—}002343) sequences of human lactoferrin; the amino acid (Accession Numbers NP_{—}851341 and CAA38572) and mRNA (Accession Numbers X54801 and NM_{—}180998) sequences of bovine lactoferrin; the amino acid (Accession Numbers JC2323, CAA55517 and AAA97958) and mRNA (Accession Number U53857) sequences of goat lactoferrin; the amino acid (Accession Number CAA09407) and mRNA (Accession Number AJ010930) sequences of horse lactoferrin; the amino acid (Accession Numbers NP_{—}999527, AAL40161 and AAP70487) and mRNA (Accession Number NM_{—}214362) sequences of pig lactoferrin; the amino acid (Accession Number NP_{—}032548) and mRNA (Accession Number NM_{—}008522) sequences of mouse lactoferrin; the amino acid (Accession Number CAA06441) and mRNA (Accession Number AJ005203) sequences of water buffalo lactoferrin; and the amino acid (Accession Number CAB53387) and mRNA (Accession Number AJ131674) sequences of camel lactoferrin. These sequences may be used according to the invention in wild type or variant form. Polypeptides encoded by these sequences may be isolated from a natural source, produced as recombinant proteins or produced by organic synthesis, using known techniques.

Methods for generating useful polypeptides and variants are known in the art and discussed below. Useful recombinant lactoferrin polypeptides and fragments and methods of producing them are reported in US patent specifications U.S. Pat. No. 5,571,691, U.S. Pat. No. 5,571,697, U.S. Pat. No. 5,571,896, U.S. Pat. No. 5,766,939, U.S. Pat. No. 5,849,881, U.S. Pat. No. 5,849,885, U.S. Pat. No. 5,861,491, U.S. Pat. No. 5,919,913, U.S. Pat. No. 5,955,316, U.S. Pat. No. 6,066,469, U.S. Pat. No. 6,080,599, U.S. Pat. No. 6,100,054, U.S. Pat. No. 6,111,081, U.S. Pat. No. 6,228,614, U.S. Pat. No. 6,277,817, U.S. Pat. No. 6,333,311, U.S. Pat. No. 6,455,687, U.S. Pat. No. 6,569,831, U.S. Pat. No. 6,635,447, US 2005-0064546 and US 2005-0114911.

Useful variants also include bovine lactoferrin variants bLf-a and bLf-b (Tsuji, et al. (1989); Yoshida, et al. (1991)). Further useful variants include glycosylated and aglycosyl forms of lactoferrin (Pierce, et al. (1991); Metz-Boutigue, et al. (1984); van Veen, et al. (2004)) and glycosylation mutants (having variant points of glycosylation or variant glycosyl side chains).

Useful fragments include the N-lobe and C-lobe fragments (Baker, et al., 2002) and any other lactoferrin polypeptides that retain a lactoferrin binding pocket, such as truncated lactoferrin polypeptides. Other lactoferrin fragments are described in published international patent application WO2007/043900 that is incorporated herein by reference.

Useful truncated lactoferrin polypeptides include polypeptides truncated by about 1 to about 300 amino acids, preferably about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 amino acids or more, and including polypeptides truncated at the N-terminus, at the C-terminus or at both the N-terminus and C-terminus, provided that the truncated polypeptide retains at least one of the N-lobe or the C-lobe metal ion-binding pockets. It is reported that residues Asp 60, Tyr 92, Tyr 192, and His 253 of bovine lactoferrin (without the signal sequence) are the amino acid metal ion ligands in the N-lobe. It is reported that residues Asp 395, Tyr 433, Tyr 526, and His 595 of bovine lactoferrin (without the signal sequence) are the amino acid metal ion ligands in the C-lobe. (Karthikeyan, et al., 1999)

Candidate variants or fragments of lactoferrin for use according to the present invention may be generated by techniques including but not limited to techniques for mutating wild type proteins (see Sambrook, et al. (1989) and elsewhere for a discussion of such techniques) such as but not limited to site-directed mutagenesis of wild type lactoferrin and expression of the resulting polynucleotides; techniques for generating expressible polynucleotide fragments such as PCR using a pool of random or selected primers; techniques for full or partial proteolysis or hydrolysis of wild type or variant lactoferrin polypeptides; and techniques for chemical synthesis of polypeptides. Variants or fragments of lactoferrin may be prepared by expression as recombinant molecules from lactoferrin DNA or RNA, or variants or fragments thereof. Nucleic acid sequences encoding variants or fragments of lactoferrin may be inserted into a suitable vector for expression in a cell, including eukaryotic cells such as but not limited to Aspergillus or bacterial cells such as but not limited to E. coli. Lactoferrin variants or fragments may be prepared using known PCR techniques including but not limited to error-prone PCR and DNA shuffling. Error-prone PCR is a process for performing PCR under conditions where the copying fidelity of the DNA polymerase is low, such that a high rate of point mutations is obtained along the entire length of the PCR product (Leung; et al. (1989); Cadwell, et al. (1992)). DNA shuffling refers to forced homologous recombination between DNA molecules of different but highly related DNA sequence in vitro, caused by random fragmentation of the DNA molecule based on sequence homology, followed by fixation of the crossover by primer extension in a PCR reaction (Stemmer (1994)). Suitable lactoferrin nucleic acid sequences for use in such methods include those listed above or may be generated by known methods including, for example, reverse transcription-PCR (RT-PCR) of tissue RNA isolates. Suitable primers for RT-PCR may be designed with reference to the mRNA sequences listed above. Commercial kits are available for RT-PCR (for example, Cells-to-cDNA™ kits from Ambion, USA).

Variants or fragments of lactoferrin may also be generated by known synthetic methods (see Kimmerlin, et al., 2005, for example).

Metal ion-binding variants or fragments of lactoferrin may be obtained by known techniques for isolating metal-binding polypeptides including but not limited to metal affinity chromatography, for example. Candidate variants or fragments of lactoferrin may be contacted with free or immobilised metal ions, such as Fe³⁺ and purified in a suitable fashion. For example, candidate variants or fragments may be contacted at neutral pH with a metal ion immobilised by chelation to a chromatography matrix comprising iminodiacetic acid or tris(carboxymethyl)ethylenediamine ligands. Bound variants or fragments may be eluted from the supporting matrix and collected by reducing the pH and ionic strength of the buffer employed. Metal-bound variants or fragments may be prepared according to the methods described above and below and described in the Examples below.

Functional variants, fragments and hydrolysates of lactoferrin may be obtained by selecting variants, fragments and hydrolysates of lactoferrin and assessing their efficacy in methods of the present invention by employing the methodologies set out in the Examples described below.

In one embodiment the lactoferrin is any mammalian lactoferrin including but not limited to sheep, goat, pig, mouse, water buffalo, camel, yak, horse, donkey, llama, bovine or human lactoferrin. Preferably the lactoferrin is bovine lactoferrin.

In another embodiment the lactoferrin is any recombinant mammalian lactoferrin including but not limited to recombinant sheep, goat, pig, mouse, water buffalo, camel, yak, horse, donkey, llama, bovine or human lactoferrin. Preferably the lactoferrin is recombinant bovine lactoferrin. Recombinant lactoferrin May be produced by expression in cell free expression systems or in transgenic animals, plants, fungi or bacteria, or other useful species. Alternatively, lactoferrin may be produced using known organic synthetic methods.

In yet another embodiment the lactoferrin is isolated from milk, preferably sheep, goat, pig, mouse, water buffalo, camel, yak, horse, donkey, llama, bovine or human milk. Preferably the lactoferrin is isolated from milk by cation exchange chromatography followed by ultrafiltration and diafiltration.

### 3. Isolation of Lactoferrin from Milk

The following is an exemplary procedure for isolating lactoferrin from bovine milk. Fresh skim milk (7 L, pH 6.5) is passed through a 300 ml column of S Sepharose Fast Flow equilibrated in milli Q water, at a flow rate of 5 ml/min and at 4° C. Unbound protein is washed through with 2.5 bed volumes of water and bound protein eluted stepwise with approximately 2.5 bed volumes each of 0.1 M, 0.35 M, and 1.0 M sodium chloride. Lactoferrin eluting as a discreet pink band in 1 M sodium chloride is collected as a single fraction and dialysed against milli Q water followed by freeze-drying. The freeze-dried powder is dissolved in 25 mM sodium phosphate buffer, pH 6.5 and subjected to rechromatography on S Sepharose Fast Flow with a sodium chloride gradient to 1 M in the above buffer and at a flow rate of 3 ml/min. Fractions containing lactoferrin of sufficient purity as determined by gel electrophoresis and reversed phase HPLC are combined, dialyzed and freeze-dried. Final purification of lactoferrin is accomplished by gel filtration on Sephacryl 300 in 80 mM dipotassium phosphate, pH 8.6, containing 0.15 M potassium chloride. Selected fractions are combined, dialyzed against milli Q water, and freeze-dried. The purity of this preparation is greater than 95% as indicated by HPLC analysis.

### 4. Metal Ion Saturation or Depletion of Lactoferrin

Iron saturation is achieved by addition of a 2:1 molar excess of 5 mM ferric nitrilotriacetate (Foley and Bates (1987)) to a 1% solution of the purified lactoferrin in 50 mM Tris, pH 7.8 containing 10 mM sodium bicarbonate. Excess ferric nitrilotriacetate is removed by dialysis against 100 volumes of milli Q water (twice renewed) for a total of 20 hours at 4° C. The iron-loaded (holo-) lactoferrin may then be freeze-dried. Varying degrees of iron saturation may be obtained by providing less of the metal ion donor, as described in the examples below. Another method of preparing metal ion lactoferrin is reported in published international patent application WO 2006/132553 that is hereby incorporated by reference. A method of maintaining or improving the keeping quality of a metal ion lactoferrin composition is reported in published international patent application WO 2006/096073 that is hereby incorporated by reference.

Iron-depleted (apo-) lactoferrin is prepared by dialysis of a 1% solution of the highly purified lactoferrin sample in water against 30 volumes of 0.1 M citric acid, pH 2.3, containing 500 mg/L disodium EDTA, for 30 h at 4° C. (Masson and Heremans (1966)). Citrate and EDTA are then removed by dialysis against 30 volumes of milli Q water (once renewed) and the resulting colourless solution may be freeze-dried.

A lactoferrin polypeptide can contain an iron ion (as in a naturally occurring lactoferrin polypeptide) or a non-iron metal ion (e.g., a copper ion, a chromium ion, a cobalt ion, a manganese ion, or a zinc ion). For instance, lactoferrin isolated from bovine milk can be depleted of iron and then loaded with another type of metal ion. For example, copper loading can be achieved according to the same method for iron loading described above. For loading lactoferrin with other metal ions, the method of Ainscough, et al. (1979) can be used.

In one embodiment the metal ion is an ion selected from the group comprising aluminium, copper, chromium, cobalt, gold, iron, manganese, platinum, ruthenium and zinc ions. Preferably the metal ion is an iron ion.

In a preparation of a composition for use according to the invention, a lactoferrin polypeptide or metal ion-binding lactoferrin fragment can be of a single species, or of different species. For instance, the polypeptides or fragments can each contain a different number of metal ions or a different species of metal ions; or the lengths of the polypeptides can vary, e.g., some are full-length polypeptides and some are fragments, and the fragments can each represent a particular portion of a full-length polypeptide. Such a preparation can be obtained from a natural source or by mixing different lactoferrin polypeptide species. For example, a mixture of lactoferrin polypeptides of different lengths can be prepared by proteinase digestion (complete or partial) of full-length lactoferrin polypeptides. The degree of digestion can be controlled according to methods well known in the art, e.g., by manipulating the amount of proteinase or the time of incubation, and described below. A full digestion produces a mixture of various fragments of full-length lactoferrin polypeptides; a partial digestion produces a mixture of full-length lactoferrin polypeptides and various fragments.

### 5. Preparation of Lactoferrin Fragments or Lactoferrin Hydrolysates

Useful lactoferrin fragments are described in published international patent applications WO 2006/054908 and WO 2007/043900. Hydrolysates containing candidate functional fragments can be prepared by selecting suitable enzymes with known specificity of cleavage, such as trypsin or chymotrypsin, and controlling/limiting proteolysis by pH, temperature, time of incubation and enzyme to substrate ratio. Refinement of such isolated peptides can be made using specific endopeptidases. As an example, bovine lactoferricin can be produced by cleavage of bovine lactoferrin with pepsin at pH 2.0 for 45 min at 37° C. (Facon & Skura, 1996), or at pH 2.5, 37° C. for 4 h using enzyme at 3% (w/w of substrate) (Tomita et al., 1994). The peptide can then be isolated by reversed phase HPLC (Tomita et al., 1994) or hydrophobic interaction chromatography (Tomita et al., 2002). In one embodiment hydrolysates useful herein contain one or more functional fragments.

Alternatively, lactoferrin peptides can be produced by well established synthetic Fmoc chemistry as described for human kaliocin-1 (NH2-FFSASCVPGADKGQFPNLCRLCAGTGENKCA-COOH) and the lactoferricin derived peptide (NH2-TKCFQWQRNMRKVRGPPVSCIKR-COOH) in Viejo-Diaz et al., (2003); and bovine lactoferricin peptide (NH2-RRWQWRMIKKLG-COOH) as described in Nguyen et al., (2005); and lactoferrampin (NH2-WKLLSKAQEKFGKNKSR-COOH) and shorter fragments as described in van der Kraan et al., (2004).

In general, SDS-PAGE may be used to estimate the degree of hydrolysis by comparison of the hydrolysate to a molecular weight standard Size exclusion chromatography may be used to separate various species within a hydrolysate and to estimate a molecular weight distribution profile.

In a preferred hydrolytic method, bovine lactoferrin was dissolved to 20 mg/mL in 50 mM Tris pH 8.0, 5 mM CaCl2. Trypsin (Sigma T8642, TPCK treated, Type XII from bovine pancreas, 11700 U/mg protein) was added at an enzyme substrate ratio of 1:50 w/w and the mixture incubated at 25° C. for 3 h. The reaction was stopped by the addition of PMSF to 1 mM final concentration and extent of digestion monitored by SDS-PAGE. The tryptic digest (4 mL) was applied to gel filtration on Sephacryl S300 (Amersham GE) (90 cm×2.6 cm column) in 50 mM Tris, 0.15M NaCl pH 8.0. Suitable fractions containing the major fragments of bovine lactoferrin (Legrand et al., 1984) were then subjected to cation exchange chromatography on S Sepharose fast Flow (Amersham GE) (15 cm×1.6 cm column) using sodium phosphate buffer pH 6.5 and a salt gradient to 1 M NaCl. Final separation of the C lobe and N+C lobes was achieved by further gel filtration on Sephacryl S300 as above but using 10% v/v acetic acid as eluent (Mata et al., 1994). The identity of the dialysed (versus milli-Q water) and freeze-dried fragments was confirmed by SDS-PAGE and Edman N-terminal sequencing.

In another method, a tryptic digest as above was separated by RP-HPLC on a Vydac C18 column as in Superti et al., (2001) and the high mass fragments corresponding to C-lobe and N-lobe fragments recovered. Identity was confirmed by MALDI MS.

### 6. Anti-Tumour Food Factors

Anti-tumour food components are reviewed in Park, et al., 2002 and Kris-Etherton, 2002. In one embodiment the anti-tumour food factor is selected from vitamin D (including vitamin D1 [lumisterol], vitamin. D2 [calciferol or ergocalciferol], vitamin D3 [cholecalciferol], vitamin D4 [22-dihydroerogocalciferol] and vitamin D5 [sitocalciferol]), vitamin D analogues (including but not limited to those referenced below), soy protein, one or more soybean components (including those selected from the group comprising but not limited to omega-3 fatty acids from soy, isoflavones from soy (e.g. genistein and/or daidzein), and lunasin peptides (such as those described in U.S. Pat. No. 6,107,287 and U.S. Pat. No. 6,544,956 that are incorporated herein by reference, and those having accession numbers AAE49016, AAE49017, AAP62458 and AAP62459), polyphenols (from green or black tea for example), lycopene (or tomato juice for example), wheat bran, flavonoids (or apple juice for example), inositol, resveratrol (or grape juice for example), propolis, mushroom extract, anthocyanins (or berry juice for example), almonds, ginseng, and casein hydrolysate.

Examples of vitamin D compounds useful herein include but are not limited to calcitriol (1-alpha,25-dihydroxy [1,25(OH)2D3]; 1,25-dihydroxycholecalciferol), 1,25-dihydroxyergocalciferol, calcifediol (25-hydroxycholecalciferol), 25-hydroxyergocalciferol, ergocalciferol (and its precursor ergosterol), cholecalciferol (and its precursor 7-dehydrocholesterol), doxercalciferol, dihydrotachysterol, paracalcitol, seocalcitol [EB 1089; 1(S),3(R)-Dihydroxy-20(R)-(5′-ethyl-5′-hydroxyhepta-1′(E),3′(E)-dien-1′-yl)-9,10-secopregna-5(Z),7(E),10(19)-triene)] as well as derivatives, analogues, homologs, precursors and metabolites thereof.

In one embodiment the anti-tumour food factor is selected from the group comprising anti-tumour foods and anti-tumour food components. In one embodiment the anti-tumour food may be a functional food or derivative thereof that has anti-cancerous properties including fruits, vegetables, legumes, nuts, seeds, grains, spices, herbs, fungi, probiotics, apples, apricots, beans (eg green bean, black bean), chick peas, berries (eg blueberries, raspberries), cruciferous vegetables (eg broccoli, brussel sprouts, cabbage, cauliflower, collards, kale, kohlrabi, bok choy, radish, mustards, and turnips), carrot, cheese, corn products, cranberries, egg plant, flaxseed, allium vegetables [eg garlic, onion, spring onion (scallions), chive, leek, shallot], ginger (including ginger components gingerol, paradol, and beta-elemene); ginseng, grapefruit, grapes, grape juice, green or black tea, horseradish, kiwifruit, kumara, leeks, lemons, limes, noni fruit, onions, oranges, peanuts, peppers, rye products, salmon, soy milk products, soy nuts, soybeans, squash, tangerines, tomatoes, wheat bran products, rice, papaya, pawpaw, peaches, persimmons, strawberries, taro leaves, green banana, mango, watercress, yams, and almonds.

In one embodiment the anti-tumour food component is selected from the group comprising soy protein, one or more soybean components (including those selected from the group comprising but not limited to omega-3 fatty acids from soy, isoflavones from soy (e.g. genistein and/or daidzein), and lunasin peptides (such as those described in U.S. Pat. No. 6,107,287 and U.S. Pat. No. 6,544,956 that are incorporated herein by reference, and those having accession numbers AAE49016, AAE49017, AAP62458 and AAP62459), shark cartilage, garlic extracts, selenium supplementation, tea extracts (e.g. green or black tea polyphenols/catechins/epigallocatechin gallate), curcuminoids, caffeine, carnosic acid, capsaicin, sesquiterpene lactones (eg parthenolide, costunolide, yomogin), cotylenin A, humulone, arginine, glutamine, retinoids from green leaf vegetables, cocoa powder, lycopene, glucosinolates from cruciferous vegetables, organosulphur compounds (allicin, diallyl sulfide, diallyl disulfide, allyl mercaptan), N-acetyl cysteine, allium compounds, carotenoids (including but not limited to beta-carotene), coumarins, dietary fibres, dithiolthiones, flavonoids (eg myricetin, quercetin, rutin), indoles, inositol, inositol hexaphosphate, isoflavones (genistein, daidzein), isothiocyanates, monoterpenes (eg limonene, perillic acid, methol, carveol), wheat bran, diterpene esters, polyphenols, riboflavin 5′ phosphate, cinnamaldehyde, vanillin, umbelliferone, phenols (eg cinnamic acid), polyphenols, plant sterols (eg sitostanol, stigmasterol, campesterol), acylglycosylsterols, phytosteroids, protease inhibitors, saponins, isoprenoids, terprenoids, tocotrienols, retinoids, ellagic acid, polyamines, resveratrol, hydroxycinnamic acids [eg (E)-ferulic acid and (E)-p-coumaric acid], chlorophyllin, propolis and some of its components (eg caffeic acid, phenyl esters, artellipin C), red wine, tannic acid, mushroom extracts, anthocyanins (eg cyanidins), mushroom beta-glucans (eg lentinan), spinach leaf extracts, natural antioxidant mixture from spinach, leaf, noni juice, vitamins A, B6, C, and E, extract of Siamese cassia, extract of Beta vulgaris, extracts of lemon grass and bamboo grass, carnosic acid, capsaicin, sesquiterpene lactones (eg parthenolide, costunolide, yomogin), cotylenin A, humulone, and omega-3 fatty acids (including eicosapentaenoic acid (EPA) and docasahexaenoic acid (DHA)), and combinations thereof.

In one embodiment the anti-tumour food component is selected from the group comprising vitamin D, vitamin B6, taurine, arginine, glutamine, alpha-lactalbumin, colostrum whey, full or partial casein hydrolysates, casein peptide(s) known to be immunostimulatory (eg immunocasokinins, caseinophosphopeptides, casomorphins, casokinins), colostrinin peptide, colostrum, calcium and calcium phosphate, folate, cysteine-rich milk proteins, lactoperoxidase, HAMLET (alpha-lactalbumin-oleic acid complex), fragments of plasminogen, prosaposin, saposins, catalase, lactoperoxidase, fatty acid binding protein, ribonuclease, beta-glucuronidase inhibitor, BRCA1, BRCA2, CD36, interferon, tumour necrosis factor, interleukin 2 (IL-2), kininogen and fragments, kininostatin, cystatin, fetuin, neutrophil defensins, interleukin 12 (IL-12), chitinase-like proteins, dystroglycan, prostasin, SPARC-like proteins, and thrombospondin, or a combination thereof.

### Soy Protein

Soy has been promoted as an agent that aids heart health and healthy bones, prevents cancer and alleviates menopausal symptoms (Kerwin, 2004). The anti-cancer effects of soy have been attributed to soy protein itself which is lower in sulphur amino acid content than animal protein and has been shown to inhibit the development of carcinogen-induced tumors in animals. Other soy components with anti-cancer activity include protease inhibitors, isoflavones such as genistein which can have anti-cancer or pro-cancer effects, and saponins. One embodiment employs soy, soy protein, soy protein concentrate, soy protein isolate, soy milk, soy yoghurt, soy cheese, soy nuts, soybeans, soybean meal, soybean flour, or soy butter.

### Vitamin D and Vitamin D Receptor Ligands

Vitamin D is widely reported as having anti-cancer properties (Harris, et al., 2004). In addition, vitamin D is believed to decrease the risk of developing many common and serious diseases apart from cancer, including type 1 diabetes, multiple sclerosis, cardiovascular disease, and osteoporosis (Holick, 2004). Dairy products are a major dietary source of vitamin D in countries such as New Zealand where milk and other dairy products are fortified with vitamin D. Vitamin D3 is 25-hydroxylated in the liver and converted in the kidney and peripheral organs into the active hormonal form 1-alpha,25-dihydroxy [1,25(OH)2D3] (calcitriol), which affects multiple processes related to cell growth and development. It exerts antiproliferative effects on tumour cells by causing arrest in the G0/G1 phase of the cell cycle, which leads to the down-regulation of growth promoting factors such as IGF-1, and the upregulation of negative growth regulators such as transforming growth factor beta. It causes tumour apoptosis, and is an inhibitor of tumour angiogenesis and metastasis (Nakagawa, et al., 2005). Administration of the active 1,25(OH)2D3 metabolite at doses necessary to inhibit tumor growth are associated with hypercalcemic toxicity. Synthetic structural analogues of 1,25(OH)2D3 that do not induce hypercalcemia have been reported to inhibit tumour growth and induce regression of tumors in animal models (Colston, et al., 2003; Nolan, et al., 1998; van Weelden, et al., 1998). The anti-cancer activity of vitamin D receptor (VDR) ligands has been demonstrated in models of carcinoma of the bladder, breast, colon, endometrium, kidney, lung, pancreas, prostate, sarcomas of the soft tissues and bone, neuroblastoma, glioma, melanoma, squamous cell carcinoma (SCC), and others (Beer, et al., 2004).

Phase I trials have demonstrated that intermittent weekly dosing allows substantial dose escalation and has produced potentially therapeutic peak calcitriol concentrations (Beer, et al., 2001). A phase II study reported encouraging levels of anti-tumour activity for the combination of high-dose 1-alpha,25-dihydroxyvitamin D and docetaxel administered on a weekly schedule in patients with androgen-independent prostate cancer (Beer, et al., 2003). Numerous studies have shown that daily intakes of 10,000 IU in humans are safe (in the absence of sunshine). Most cases of vitamin. D toxicity have been reported to occur after the ingestion of greater than 50,000 IU daily for several years. Some argue that vitamin D is not particularly toxic (Saul, 2003).

Compounds that act as vitamin D receptor (VDR) ligands and that may be useful herein are generally grouped into three classes: (1) “deltanoids” that have a secosteroid scaffold, (2) “pseudo-secosteroids” that have the A-ring of vitamin D but one of the C or D rings is broken, and (3) “non-secosteroids” that are structurally distinct from secosteroids. For reviews of useful vitamin D analogues see Guyton, et al., 2003; Guyton, et al., 2001; Peleg, et al., 2003; and Yee, et al., 2005.

Useful vitamin D analogues are described in U.S. Pat. No. 3,822,254, U.S. Pat. No. 3,856,780, U.S. Pat. No. 3,901,928, U.S. Pat. No. 3,994,878, U.S. Pat. No. 4,021,423, U.S. Pat. No. 4,026,882, U.S. Pat. No. 4,028,349, U.S. Pat. No. 4,038,272, U.S. Pat. No. 4,069,321, U.S. Pat. No. 4,159,326, U.S. Pat. No. 4,225,525, U.S. Pat. No. 4,263,215, U.S. Pat. No. 4,265,822, U.S. Pat. No. 4,269,777, U.S. Pat. No. 4,284,577, U.S. Pat. No. 4,292,250, U.S. Pat. No. 4,297,289, U.S. Pat. No. 4,307,231, U.S. Pat. No. 4,310,467, U.S. Pat. No. 4,351,767, U.S. Pat. No. 4,360,470, U.S. Pat. No. 4,388,243, U.S. Pat. No. 4,407,754, U.S. Pat. No. 4,448,726, U.S. Pat. No. 4,456,553, U.S. Pat. No. 4,512,925, U.S. Pat. No. 4,517,125, U.S. Pat. No. 4,554,105, U.S. Pat. No. 4,613,594, U.S. Pat. No. 4,661,294, U.S. Pat. No. 4,666,634, U.S. Pat. No. 4,670,190, U.S. Pat. No. 4,717,721, U.S. Pat. No. 4,769,181, U.S. Pat. No. 4,772,433, U.S. Pat. No. 4,804,502, U.S. Pat. No. 4,832,875, U.S. Pat. No. 4,847,012, U.S. Pat. No. 4,851,400, U.S. Pat. No. 4,851,401, U.S. Pat. No. 4,853,378, U.S. Pat. No. 4,857,518, U.S. Pat. No. 4,866,048, U.S. Pat. No. 4,891,364, U.S. Pat. No. 4,892,821, U.S. Pat. No. 4,897,387, U.S. Pat. No. 4,898,855, U.S. Pat. No. 4,929,610, U.S. Pat. No. 4,973,721, U.S. Pat. No. 4,997,824, U.S. Pat. No. 5,030,626, U.S. Pat. No. 5,030,772, U.S. Pat. No. 5,035,783, U.S. Pat. No. 5,036,061, U.S. Pat. No. 5,039,671, U.S. Pat. No. 5,047,564, U.S. Pat. No. 5,053,401, U.S. Pat. No. 5,063,221, U.S. Pat. No. 5,069,905, U.S. Pat. No. 5,075,465, U.S. Pat. No. 5,086,191, U.S. Pat. No. 5,087,619, U.S. Pat. No. 5,093,519, U.S. Pat. No. 5,098,899, U.S. Pat. No. 5,110,924, U.S. Pat. No. 5,116,573, U.S. Pat. No. 5,117,018, U.S. Pat. No. 5,145,846, U.S. Pat. No. 5,149,846, U.S. Pat. No. 5,157,135, U.S. Pat. No. 5,175,217, U.S. Pat. No. 5,185,150, U.S. Pat. No. 5,190,935, U.S. Pat. No. 5,194,248, U.S. Pat. No. 5,194,431, U.S. Pat. No. 5,200,536, U.S. Pat. No. 5,202,266, U.S. Pat. No. 5,206,229, U.S. Pat. No. 5,206,230, U.S. Pat. No. 5,210,237, U.S. Pat. No. 5,225,579, U.S. Pat. No. 5,232,836, U.S. Pat. No. 5,237,110, U.S. Pat. No. 5,246,925, U.S. Pat. No. 5,247,104, U.S. Pat. No. 5,247,123, U.S. Pat. No. 5,260,290, U.S. Pat. No. 5,274,142, U.S. Pat. No. 5,278,155, U.S. Pat. No. 5,281,731, U.S. Pat. No. 5,283,345, U.S. Pat. No. 5,292,728, U.S. Pat. No. 5,292,977, U.S. Pat. No. 5,316,770, U.S. Pat. No. 5,321,018, U.S. Pat. No. 5,328,903, U.S. Pat. No. 5,342,833, U.S. Pat. No. 5,342,975, U.S. Pat. No. 5,362,719, U.S. Pat. No. 5,366,736, U.S. Pat. No. 5,371,249, U.S. Pat. No. 5,373,004, U.S. Pat. No. 5,374,629, U.S. Pat. No. 5,376,651, U.S. Pat. No. 5,378,695, U.S. Pat. No. 5,380,720, U.S. Pat. No. 5,387,582, U.S. Pat. No. 5,389,622, U.S. Pat. No. 5,391,755, U.S. Pat. No. 5,393,900, U.S. Pat. No. 5,395,829, U.S. Pat. No. 5,397,776, U.S. Pat. No. 5,401,731, U.S. Pat. No. 5,401,732, U.S. Pat. No. 5,403,832, U.S. Pat. No. 5,403,940, U.S. Pat. No. 5,411,949, U.S. Pat. No. 5,414,098, U.S. Pat. No. 5,428,029, U.S. Pat. No. 5,430,196, U.S. Pat. No. 5,446,034, U.S. Pat. No. 5,446,035, U.S. Pat. No. 5,446,225, U.S. Pat. No. 5,447,924, U.S. Pat. No. 5,449,668, U.S. Pat. No. 5,451,574, U.S. Pat. No. 5,457,217, U.S. Pat. No. 5,459,136, U.S. Pat. No. 5,486,636, U.S. Pat. No. 5,488,120, U.S. Pat. No. 5,494,905, U.S. Pat. No. 5,502,224, U.S. Pat. No. 5,508,392, U.S. Pat. No. 5,512,554, U.S. Pat. No. 5,516,525, U.S. Pat. No. 5,525,745, U.S. Pat. No. 5,532,228, U.S. Pat. No. 5,532,391, U.S. Pat. No. 5,536,713, U.S. Pat. No. 5,545,633, U.S. Pat. No. 5,552,392, U.S. Pat. No. 5,554,599, U.S. Pat. No. 5,561,123, U.S. Pat. No. 5,563,286, U.S. Pat. No. 5,565,442, U.S. Pat. No. 5,565,589, U.S. Pat. No. 5,578,587, U.S. Pat. No. 5,581,006, U.S. Pat. No. 5,583,125, U.S. Pat. No. 5,585,368, U.S. Pat. No. 5,585,369, U.S. Pat. No. 5,587,497, U.S. Pat. No. 5,589,471, U.S. Pat. No. 5,597,932, U.S. Pat. No. 5,599,958, U.S. Pat. No. 5,612,325, U.S. Pat. No. 5,612,326, U.S. Pat. No. 5,612,328, U.S. Pat. No. 5,616,744, U.S. Pat. No. 5,618,805, U.S. Pat. No. 5,629,302, U.S. Pat. No. 5,633,241, U.S. Pat. No. 5,637,742, U.S. Pat. No. 5,654,292, U.S. Pat. No. 5,661,140, U.S. Pat. No. 5,663,157, U.S. Pat. No. 5,665,716, U.S. Pat. No. 5,686,435, U.S. Pat. No. 5,691,328, U.S. Pat. No. 5,700,791, U.S. Pat. No. 5,710,142, U.S. Pat. No. 5,710,294, U.S. Pat. No. 5,716,945, U.S. Pat. No. 5,719,297, U.S. Pat. No. 5,721,224, U.S. Pat. No. 5,721,225, U.S. Pat. No. 5,747,478, U.S. Pat. No. 5,747,479, U.S. Pat. No. 5,750,517, U.S. Pat. No. 5,750,746, U.S. Pat. No. 5,753,638, U.S. Pat. No. 5,756,489, U.S. Pat. No. 5,756,733, U.S. Pat. No. 5,763,426, U.S. Pat. No. 5,763,428, U.S. Pat. No. 5,763,429, U.S. Pat. No. 5,767,111, U.S. Pat. No. 5,786,347, U.S. Pat. No. 5,789,399, U.S. Pat. No. 5,804,573, U.S. Pat. No. 5,804,574, U.S. Pat. No. 5,811,414, U.S. Pat. No. 5,811,562, U.S. Pat. No. 5,817,648, U.S. Pat. No. 5,827,883, U.S. Pat. No. 5,840,938, U.S. Pat. No. 5,843,927, U.S. Pat. No. 5,843,928, U.S. Pat. No. 5,847,173, U.S. Pat. No. 5,856,317, U.S. Pat. No. 5,856,536, U.S. Pat. No. 5,869,472, U.S. Pat. No. 5,872,113, U.S. Pat. No. 5,872,140, U.S. Pat. No. 5,877,168, U.S. Pat. No. 5,880,113, U.S. Pat. No. 5,880,114, U.S. Pat. No. 5,883,271, U.S. Pat. No. 5,888,994, U.S. Pat. No. 5,902,806, U.S. Pat. No. 5,905,074, U.S. Pat. No. 5,919,986, U.S. Pat. No. 5,929,056, U.S. Pat. No. 5,932,565, U.S. Pat. No. 5,935,624, U.S. Pat. No. 5,936,105, U.S. Pat. No. 5,936,133, U.S. Pat. No. 5,939,406, U.S. Pat. No. 5,939,408, U.S. Pat. No. 5,945,410, U.S. Pat. No. 5,962,707, U.S. Pat. No. 5,972,917, U.S. Pat. No. 5,981,779, U.S. Pat. No. 5,981,780, U.S. Pat. No. 5,986,112, U.S. Pat. No. 5,994,332, U.S. Pat. No. 6,008,209, U.S. Pat. No. 6,013,814, U.S. Pat. No. 6,017,907, U.S. 6,017,908, U.S. Pat. No. 6,025,346, U.S. Pat. No. 6,028,208, U.S. Pat. No. 6,030,962, U.S. Pat. No. 6,034,074, U.S. Pat. No. 6,040,300, U.S. Pat. No. 6,040,461, U.S. Pat. No. 6,043,385, U.S. Pat. No. 6,043,386, U.S. Pat. No. 6,071,897, U.S. Pat. No. 6,072,062, U.S. Pat. No. 6,075,015, U.S. Pat. No. 6,080,878, U.S. Pat. No. 6,100,294, U.S. Pat. No. 6,103,709, U.S. Pat. No. 6,114,317, U.S. Pat. No. 6,121,312, U.S. Pat. No. 6,121,469, U.S. Pat. No. 6,124,276, U.S. Pat. No. 6,127,559, U.S. Pat. No. 6,177,586, U.S. Pat. No. 6,184,398, U.S. Pat. No. 6,197,982, U.S. Pat. No. 6,207,656, U.S. Pat. No. 6,229,030, U.S. Pat. No. 6,262,041, U.S. Pat. No. 6,262,283, U.S. Pat. No. 6,277,837, U.S. Pat. No. 6,281,249, U.S. Pat. No. 6,284,928, U.S. Pat. No. 6,288,249, U.S. Pat. No. 6,291,443, U.S. Pat. No. 6,291,444, U.S. Pat. No. 6,291,693, U.S. Pat. No. 6,291,694, U.S. Pat. No. 6,294,688, U.S. Pat. No. 6,307,075, U.S. Pat. No. 6,310,226, U.S. Pat. No. 6,316,642, U.S. Pat. No. 6,326,503, U.S. Pat. No. 6,329,357, U.S. Pat. No. 6,329,538, U.S. Pat. No. 6,331,642, U.S. Pat. No. 6,335,458, U.S. Pat. No. 6,353,123, U.S. Pat. No. 6,358,939, U.S. Pat. No. 6,359,152, U.S. Pat. No. 6,369,099, U.S. Pat. No. 6,372,233, U.S. Pat. No. 6,372,731, U.S. Pat. No. 6,372,926, U.S. Pat. No. 6,376,480, U.S. Pat. No. 6,380,408, U.S. Pat. No. 6,392,071, U.S. Pat. No. 6,395,287, U.S. Pat. No. 6,399,767, U.S. Pat. No. 6,399,797, U.S. Pat. No. 6,407,082, U.S. Pat. No. 6,410,523, U.S. Pat. No. 6,433,200, U.S. Pat. No. 6,441,207, U.S. Pat. No. 6,444,658, U.S. Pat. No. 6,118,421, U.S. Pat. No. 6,452,028, U.S. Pat. No. 6,455,714, U.S. Pat. No. 6,458,827, U.S. Pat. No. 6,479,474, U.S. Pat. No. 6,479,538, U.S. Pat. No. 6,482,812, U.S. Pat. No. 6,492,353, U.S. Pat. No. 6,503,893, U.S. Pat. No. 6,506,912, U.S. Pat. No. 6,521,608, U.S. Pat. No. 6,531,459, U.S. Pat. No. 6,531,460, U.S. Pat. No. 6,537,980, U.S. Pat. No. 6,537,981, U.S. Pat. No. 6,537,982, U.S. Pat. No. 6,538,145, U.S. Pat. No. 6,544,969, U.S. Pat. No. 6,548,489, U.S. Pat. No. 6,548,715, U.S. Pat. No. 6,555,699, U.S. Pat. No. 6,559,138, U.S. Pat. No. 6,566,353, U.S. Pat. No. 6,573,255, U.S. Pat. No. 6,573,256, U.S. Pat. No. 6,600,058, U.S. Pat. No. 6,603,031, U.S. Pat. No. 6,610,866, U.S. Pat. No. 6,613,920, U.S. Pat. No. 6,642,218, U.S. Pat. No. 6,646,143, U.S. Pat. No. 6,667,298, U.S. Pat. No. 6,680,309, U.S. Pat. No. 6,683,219, U.S. Pat. No. 6,689,766, U.S. Pat. No. 6,689,922, U.S. Pat. No. 6,696,431, U.S. Pat. No. 6,706,725, U.S. Pat. No. 6,774,251, U.S. Pat. No. 6,787,660, U.S. Pat. No. 6,806,262, U.S. Pat. No. 6,831,106, U.S. Pat. No. 6,831,183, U.S. Pat. No. 6,844,461, U.S. Pat. No. 6,858,595, U.S. Pat. No. 6,867,313, U.S. Pat. No. 6,890,914, U.S. Pat. No. 6,902,654, U.S. Pat. No. 6,924,400, U.S. Pat. No. 6,929,797, U.S. Pat. No. 6,960,573, US 2001007866, US 2001007907, US 2001014749, US 2001025036, US 2001036937, US 2001051617, US 2002006917, US 2002010163, US 2002010165, US 2002016313, US 2002019375, US 2002025950, US 2002032340, US 2002042403, US 2002045772, US 2002049344, US 2002061867, US 2002068723, US 2002068834, US 2002076442, US 2002087015, US 2002091109, US 2002094972, US 2002099039, US 2002103172, US 2002103173, US 2002111503, US 2002123638, US 2002128240, US 2002128241, US 2002136731, US 2002137731, US 2002183277, US 2002188142, US 2002193616, US 2003004144, US 2003009042, US 2003013690, US 2003013691, US 2003018194, US 2003018206, US 2003022872, US 2003022873, US 2003069212, US 2003073857, US 2003083319, US 2003092686, US 2003095937, US 2003105067, US 2003119795, US 2003125309, US 2003129194, US 2003130241, US 2003130242, US 2003149005, US 2003149006, US 2003166226, US 2003166622, US 2003171342, US 2003171605, US 2003181427, US 2003187287, US 2003188756, US 2003191093, US 2003195175, US 2003195176, US 2003195259, US 2003207810, US 2004019023, US 2004019024, US 2004023934, US 2004030167, US 2004038949, US 2004043971, US 2004053813, US 2004054204, US 2004069523, US 2004082802, US 2004108198, US 2004116724; US 2004132695, US 2004133026, US 2004167104, US 2004167105, US 2004167106, US 2004214803, US 2004224929, US 2004224930, US 2004229851, US 2005009793, US 2005009794, US 2005014211, US 2005020546, US 2005026871, US 2005026877, US 2005043281, US 2005059641, US 2005063992, US 2005064018, VS 2005065087, US 2005065088, US 2005065117, US 2005065125, US 2005065126, US 2005065127, US 2005065128, US 2005065129, US 2005065130, US 2005065131, US 2005065132, US 2005065133, US 2005065134, US 2005065180, US 2005070511, US 2005070512, US 2005070513, US 2005080058, US 2005080059, US 2005101574, US 2005101575, US 2005101576, US 2005101577, US 2005101578, US 2005113348, US 2005113349, US 2005119240, US 2005119241, US 2005119242, US 2005131242, US 2005143358, US 2005164995, US 2005182032, US 2005182034, US 2005182035, US 2005182144, US 2005192255, US 2005192256, US 2005203071, US 2005209203, US 2005227950, US 2005234009, US 2005261256 and US 2005277171. Assessment of these analogues in a method of the invention may be carried out by following the protocols described in the examples below.

### 7. Immune Enhancement

The present inventors have found that lactoferrin and at least one anti-tumour food factor are able to stimulate and therefore enhance the immune system. In particular, as shown in the examples below, lactoferrin and at least one anti-tumour food factor are able to stimulate the generation of antigen-specific cytolytic activity (the activity of immune cells, particularly cytotoxic T-lymphocytes) and/or NK cell activity, improve the cellular immune response to antigens (through the activity of at least cytotoxic T-lymphocytes), improve immune protection (by at least restoring the activity of cytotoxic T-lymphocytes and/or NK cells and enhancing cytokine production), restore immune protection (by at least restoring or stimulating the activity of cytotoxic T-lymphocytes and/or NK cell activity and enhancing cytokine production) and generate pro-inflammatory and immunoregulatory mediators (Th1 and Th2 cytokines). It is believed that any functional variant or functional fragment of lactoferrin in combination with at least one anti-tumour food factor will exhibit similar activity as a lactoferrin in combination with at least one anti-tumour food factor.

Oral iron-saturated bovine lactoferrin induced significant increases in the levels of both Th1 and Th2 cytokines within the tumour and intestine, as shown in the Examples below.

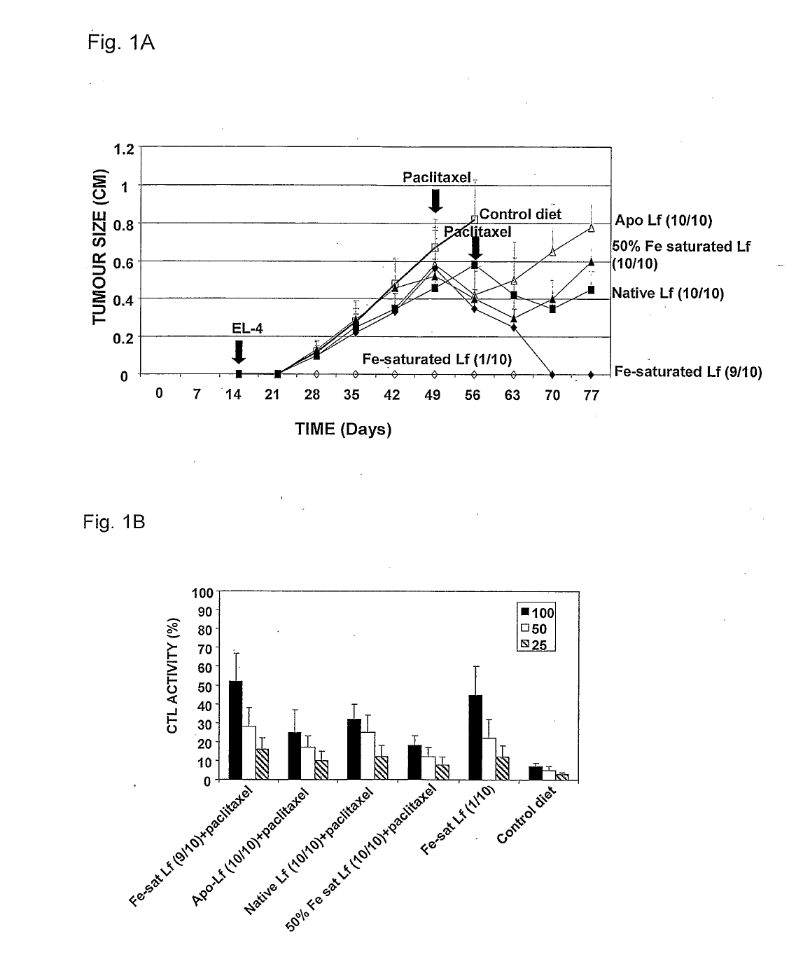

As shown in FIGS. 1A and 1B, metal ion lactoferrin is more effective than natural lactoferrin (lactoferrin having an iron saturation of less than 20%, typically 12 to 15%) for improving the generation of antigen-specific cytolytic activity and/or NK cell activity, improving the cellular immune response to antigens, improving immune protection and restoring immune protection.

Accordingly, the present invention relates to a method of stimulating the immune system of a subject comprising administration of lactoferrin and at least one anti-tumour food factor to the subject. The present invention also relates to methods of increasing the production of Th1 and Th2 cytokines within a tumor of a subject, of increasing the production of Th1 and Th2 cytokines within the intestine of a subject, of increasing the level of Th1 and Th2 cytokines in the systemic circulation of a subject, and of increasing an anti-tumour immune response in a subject.

In one embodiment of these methods of the invention, the subject is undergoing or will undergo a cancer therapy as described above. In one embodiment the subject has undergone therapy, but is in relapse or is susceptible to relapse. In one embodiment the subject has a tumour refractory to therapy with a chemotherapeutic, anti-angiogenic or immunotherapeutic agent. In one embodiment the subject has previously undergone unsuccessful therapy with a chemotherapeutic, anti-angiogenic or immunotherapeutic agent.

In one embodiment the Th1 cytokine is selected from IL-18, TNF-α and IFN-γ. In one embodiment the Th2 cytokine is selected from IL-4, IL-5, IL-6 and IL-10. In one embodiment the level of Th1 or Th2 cytokine or cytokines is increased by at least about 5, 10, 20, 30, 40, 50, 60, 70, 80, 90, 100, 150, 200, 250, 300, 350, 400, 450, 500, 550, 600, 650, 700, 750, 800, 1000, 2000′, 3000, 4000, 5000, 6000, 7000, or 8000%.

Where appropriate, these methods may be combined with treatments employing any one or more of the anti-tumour agents (including chemotherapeutic agents or immunotherapeutic agents) or anti-tumour therapies described below.

### 8. Cancer Prevention

The present inventors have found that lactoferrin in combination with at least one anti-tumour food factor is able to inhibit tumour formation and inhibit tumour growth. Lactoferrin, particularly metal ion lactoferrin releases anti-tumour factors such as T-cells and/or NK (natural killer) cells and apoptosis-inducing factors into systemic circulation, displays immune enhancing activity, anti-angiogenic activity and direct tumour cytotoxicity, and is able to induce apoptosis of tumour cells as shown in the examples below. It is believed that any functional variant or functional fragment of lactoferrin in combination with at least one anti-tumour food factor will exhibit similar activity as a lactoferrin in combination with at least one anti-tumour food factor.

The present invention has utility in preventing cancer, particularly in preventing relapse (tumour growth) after surgery such as often results from growth and proliferation of secondary tumours, preventing tumour spread after diagnosis and preparing subjects for administration of an anti-tumour agent or anti-tumour therapy.

Solid tumours must form new blood vessels before they are able to grow beyond a certain size. Therefore, inhibiting angiogenesis, particularly tumour angiogenesis (blood vessel formation to supply tumours) has clear applications in treating cancer (Dass, 2004). As shown in the Examples below, orally administered metal ion lactoferrin and at least one anti-tumour food factor are able to significantly reduce the number of vessels in tumours and significantly reduce blood flow.

Inhibiting angiogenesis also has applications in other disorders including but not limited to cardiovascular diseases (atherosclerosis and restenosis for example), chronic inflammation (rheumatoid arthritis, osteoarthritis, and Crohn's disease for example), diabetes (diabetic retinopathy), psoriasis, endometriosis, macular degeneration and adiposity. Therefore, lactoferrin or a functional variant or functional fragment thereof and at least one anti-tumour food factor have applications outside of cancer treatment and prevention.

Similarly, orally administered metal ion lactoferrin and at least one anti-tumour food factor are able to induce apoptosis of tumour cells, as shown in the Examples below.

Therefore, the present invention also relates to methods of inhibiting tumour formation in a subject, inducing apoptosis in a subject, inducing apoptosis of tumour cells in a subject, inhibiting angiogenesis in a subject and inhibiting tumour angiogenesis in a subject comprising administration of lactoferrin or a metal ion functional valiant or functional fragment thereof to the subject.

In one embodiment the subject is susceptible to cancer. In one embodiment the subject has a tumour refractory to therapy with a chemotherapeutic, anti-angiogenic or immunotherapeutic agent. In one embodiment the subject has previously undergone unsuccessful therapy with a chemotherapeutic, anti-angiogenic or immunotherapeutic agent.

Where appropriate, these methods may be combined with treatments employing any one or more of the anti-tumour agents (including chemotherapeutic agents or immunotherapeutic agents) or anti-tumour therapies described below.

### 9. Cancer Treatment and Prevention with Combination Therapies

The present inventors have found that lactoferrin and metal ion lactoferrin, preferably iron lactoferrin, preferably bovine lactoferrin, in combination with at least one anti-tumour food factor is able to inhibit tumour growth. Metal ion lactoferrin synergizes with immunotherapy (including that mediated by intratumoural gene transfer of B7-1), with chemotherapy (including with paclitaxel, doxorubicin, epirubicin or fluorouracil) or with dendritic cell therapy to substantially eradicate tumours. Metal ion lactoferrin, preferably iron lactoferrin, preferably bovine lactoferrin, is able to synergize with chemotherapy (including with paclitaxel, doxorubicin, epirubicin, fluorouracil, cyclophosphamide or methotrexate) to inhibit tumour growth. It is believed that any metal ion functional variant or functional fragment of lactoferrin in combination with at least one anti-tumour food factor will exhibit similar activity as a metal ion lactoferrin in combination with at least one anti-tumour food factor.

As described above, metal ion lactoferrin in combination with at least one anti-tumour food factor was found to release anti-tumour factors such as T-cells and/or NK (natural killer) cells and apoptosis-inducing factors into systemic circulation, display immune enhancing activity, anti-angiogenic activity and direct tumour cytotoxicity, and the ability to induce apoptosis of tumour cells as shown in the examples below. It is believed that any metal ion functional variant or functional fragment of lactoferrin in combination with at least one anti-tumour food factor will exhibit similar activity as a metal ion lactoferrin in combination with at least one anti-tumour food factor.

In one embodiment the chemotherapeutic agent is paclitaxel, doxorubicin, epirubicin, fluorouracil, cyclophosphamide or methotrexate.

In addition to the methods described above, the present invention relates to methods of inhibiting tumour growth in a subject and methods of treating or preventing cancer in a subject comprising
(a) administration of a lactoferrin or a functional variant or functional fragment thereof and at least one anti-tumour food factor, and
(b) separate, simultaneous or sequential administration of at least one anti-tumour agent or anti-tumour therapy.

In one embodiment the subject is suffering from or is susceptible to cancer. In one embodiment the subject has a tumour refractory to therapy with a chemotherapeutic, anti-angiogenic or immunotherapeutic agent. In one embodiment the subject has previously undergone unsuccessful therapy with a chemotherapeutic, anti-angiogenic or immunotherapeutic agent.

In one embodiment the at least one anti-tumour agent is administered orally or parenterally although the preferred route depends on the anti-tumor agent selected. Preferably the at least one anti-tumour agent is administered orally or by intravenous, intraperitoneal or intratumoural injection. Preferably paclitaxel, doxorubicin, epirubicin, fluorouracil, cyclophosphamide and methotrexate are administered by intravenous or intraperitoneal injection. Preferably the expression plasmid encoding B7-1 is administered by intratumoural injection. Alternatively, tumour cells can be harvested from a patient, transfected ex vivo with B7-1 expression plasmid, then transfected cells injected into a patient. Alternatively, soluble B7-Ig fusion protein can be parenterally delivered. Preferably the dendritic cell therapy is administered by intravenous, intraperitoneal, or intratumoural injection.

In one embodiment the lactoferrin is administered orally or parenterally.

In one embodiment the lactoferrin and at least one anti-tumour food factor are administered daily for at least about 1, 2, 3, 4, 5, 6, 7, 8, 9 or 10 weeks before administration of the anti-tumour agent or anti-tumour therapy. In one embodiment the lactoferrin and at least one anti-tumour food factor are administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months before administration of the anti-tumour agent or the anti-tumour therapy. In one embodiment the lactoferrin and at least one anti-tumour food factor are administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months after administration of the anti-tumour agent or the anti-tumour therapy has begun.

Preferably the lactoferrin and at least one anti-tumour food factor are administered at least once daily including continuously over a day orally, by parenteral drip or by a combination of administrative routes (oral and parenteral, for example).

In one embodiment of a method of the invention the tumour is a large tumour, as described above.

In one embodiment of a method of the invention one or both of the white blood cell count and red blood cell count of the subject is maintained or improved.

In one embodiment the lactoferrin and at least one anti-tumour food factor are administered in a dosage form comprising digestible protein, preferably casein or other protein such as other edible proteins.

In one embodiment the lactoferrin and at least one anti-tumour food factor provide a synergistic therapeutic effect that is better than the additive effects of either one alone. For example, preferably there is a greater effect on inhibition of tumour formation or growth, tumour regression, cytolytic effects, immune enhancement, generation of Th1 and Th2 cytokines, or the responsiveness of a subject or a tumour to the treatment method.

These methods may be combined with treatments employing any one or more of the anti-tumour agents (including chemotherapeutic agents or immunotherapeutic agents) or anti-tumour therapies described below.

In one embodiment the anti-tumour therapy is selected from therapies such as, but not limited to, surgery, chemotherapies, radiation therapies, hormonal therapies, biological therapies/immunotherapies, anti-angiogenic therapies, cytotoxic therapies, vaccines, nucleic acid-based vaccines (eg nucleic acids expressing a cancer antigen such as DNA vaccines including p185 vaccines), viral-based therapies (eg adeno-associated virus, lentivirus), gene therapies, small molecule inhibitor therapies, nucleotide-based therapies (eg RNAi, antisense, ribozymes etc), antibody-based therapies, oxygen and ozone treatments, embolization, and/or chemoembolization therapies.

In one embodiment the anti-tumour therapy is selected from chemotherapeutic agents including but not limited to topoisomerase inhibitor, alkylating agent (eg nitrogen mustards; ethylenimes; alkylsulfonates; triazenes; piperazines; and nitrosureas), antimetabolite (eg mercaptopurine, thioguanine, 5-fluorouracil), antibiotics [eg anthracyclines, dactinomycin, bleomycin, adriamycin, mithramycin. (topoisomerase II inhibitor, DNA strand breakage, and DNA intercalator), dactinomycin], mitotic disrupter (eg plant alkaloids like vincristine, microtubule antagonists like paclitaxel), DNA intercalating agents (eg carboplatin and cisplatin), DNA synthesis inhibitor, DNA-RNA transcription regulator, enzyme inhibitor, gene regulator or hormone response modifier, hypoxia-selective cytotoxin (eg tirapazamine), epidermal growth factor inhibitor, anti-vascular agent (eg the xanthenone 5,6-dimethylxanthenone-4-acetic acid or DMXAA; BNC105P), radiation-activated prodrug (eg nitroarylmethyl quaternary (NMQ) salts), bioreductive drugs.

In one embodiment the anti-tumour therapy is selected from chemotherapeutic agents including but not limited to irinotecan (a topoisomerase I inhibitor), cyclophosphamide (an alkylating and DNA cross-linking agent), methotrexate, fluorouracil, epirubicin and doxorubicin (an anthracycline ie a topoisomerase II inhibitor and DNA intercalator).

In one embodiment the at least one anti-tumour agent is a chemotherapeutic agent. Preferably the chemotherapeutic agent is selected from tubulin disruptors, DNA intercalators, and mixtures thereof.

Preferred tubulin disruptors include but are not limited to: taxanes such as but not limited to Paclitaxel and Docetaxel, Vinca alkaloids, Discodermolide, Epothilones A and B, Desoxyepothilone, Cryptophycins, Curacin A, Combretastatin A-4-Phosphate, BMS 247550, BMS 184476, BMS 188791; LEP, RPR 109881A, EPO 906, TXD 258, ZD 6126, Vinflunine, LU 103793, Dolastatin 10, E7010, T138067 and T900607, Colchicine, Phenstatin, Chalcones, Indanocine, T138067, Oncocidin, Vincristine, Vinblastine, Vinorelbine, Vinflunine, Halichondrin B, Isohomohalichondrin B, ER-86526, Pironetin, Spongistatin 1, Spiket P, Cryptophycin 1, Dolastatin, Cematodin, Rhizoxin, Sarcodictyin, Eleutherobin, Laulilamide, VP-16 and D-24851.

Preferred DNA intercalators include but are not limited to: Acridines, Actinomycins, Anthracyclines, Benzothiopyranoindazoles, Pixantrone, Crisnatol, Brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, carboplatin, cis-platin, actinomycin D, amsacrine, DACA, Pyrazoloacridine, Irinotecan and topotecan.

In one preferred embodiment the chemotherapeutic agent is selected from paclitaxel, doxorubicin, epirubicin, fluorouracil, cyclophosphamide and methotrexate.

In one embodiment the anti-tumour agent is an immunotherapeutic agent. Preferably the immunotherapeutic agent is an expression plasmid encoding the T cell co-stimulator B7-1, a T cell co-stimulator, or a functionally related molecule, for example a B7-Ig chimera.

In one embodiment the anti-tumour agent or therapy comprises dendritic cell therapy.

In one embodiment the anti-tumour agent comprises one or more angiogenesis inhibitors such as, but not limited to, those listed in published international patent application WO 2006/054908 that is incorporated by reference herein. Additional examples of anti-tumour agents that can be used in the various embodiments of the invention, include, but are not limited to, those listed in published international patent application WO 2006/054908 that is incorporated by reference herein. Other anti-tumour agents useful herein include, but are not limited to, those listed in published international patent application WO 2006/054908 that is incorporated by reference herein.

In one embodiment the radiation therapy includes external beam radiation therapy (including gamma-ray and x-ray therapy) and internal radiation therapy using radioisotopes. Radioisotopes may also be used as anti-tumour agents according to the invention.

### 10. Methods of Increasing Tumour Responsiveness to Therapy

The inventors have shown in the Examples below that orally administered metal ion lactoferrin and at least one anti-tumour food factor are able to increase the responsiveness of a subject and increase the sensitivity of a tumour to anti-tumour agents. It is believed that lactoferrin and any functional variant, functional fragment, metal ion functional variant or metal ion functional fragment of lactoferrin in combination with at least one anti-tumour food factor will exhibit similar activity as a metal ion lactoferrin in combination with at least one anti-tumour food factor.

Therefore, the present invention also relates to a method of increasing the responsiveness of a subject to a therapy, such as an anti-cancer therapy selected from the group comprising surgery, chemotherapies, radiation therapies, hounonal therapies (eg tamoxifen, aromatase inhibitors), biological therapies/immunotherapies, anti-angiogenic therapies, cytotoxic therapies, vaccines, nucleic acid-based vaccines (eg nucleic acids expressing a cancer antigen such as DNA vaccines including p185 vaccines), viral-based therapies (eg adeno-associated virus, lentivirus), gene therapies, small molecule inhibitor therapies, nucleotide-based therapies (eg RNAi, antisense, ribozymes etc), antibody-based therapies, oxygen and ozone treatments, embolization, and/or chemoembolization therapy and combinations thereof comprising administration of lactoferrin and at least one anti-tumour food factor to a subject in need thereof separately, simultaneously or sequentially with the therapy.

The present invention also relates, to a method of increasing the sensitivity of a tumour in a subject to a cancer therapy comprising oral or parenteral administration of lactoferrin and at least one anti-tumour food factor to a subject in need thereof separately, simultaneously or sequentially with administration of the therapy. Preferably the lactoferrin is as described above. Preferably the anti-tumour food factor is one described above. Preferably the therapy is one described above.

Equally, the present invention also relates to a method of speeding the recovery of a subject undergoing cancer therapy comprising administration of lactoferrin and at least one anti-tumour food factor to a subject in need thereof separately, simultaneously or sequentially with administration of the therapy. In this embodiment of the invention, the subject recovers from the effects of the cancer or the cancer therapy more quickly than a subject not treated according to the invention. Preferably the subject is able to reduce the dose of or time spent receiving a cancer therapy.

These methods may be combined with treatments employing any one or more of the anti-tumour agents (including chemotherapeutic agents or immunotherapeutic agents) or anti-tumour therapies described above.

In one embodiment the lactoferrin and the at least one anti-tumour food factor are administered daily for at least about 1, 2, 3, 4, 5, 6, 7, 8, 9 or 10 weeks before administration of the anti-tumour agent or anti-tumour therapy. In one embodiment the lactoferrin and the at least one anti-tumour food factor are administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months before administration of the anti-tumour agent or the anti-tumour therapy. In one embodiment the lactoferrin and the at least one anti-tumour food factor are administered for at least about 1, 2, 3, 4, 5, 6, 7, 8, 9, 10, 11, 12, 13, 14, 15, 16, 17, 18, 19, 20 or 21 days or for at least about 1, 2, 3, 4, 5, 6, 7 or 8 weeks or for at least about 1, 2, 3, 4, 5 or 6 months after administration of the anti-tumour agent or the anti-tumour therapy has begun.

### 11. Tumour Types

In one embodiment the tumour is, the tumour cells are or the cancer is a leukemia, colon carcinoma, breast cancer, melanoma, skin or lung cancer. Tumour types to which the present invention relates are listed in published international patent application WO 2006/054908 that is incorporated by reference herein.

In one embodiment the tumour is, the tumour cells are or the cancer is a leukemia such as but not limited to, acute leukemia, acute lymphocytic leukemia, acute granulocytic leukemia, acute myelocytic leukemia such as myeloblastic, promyelocytic, myelomonocytic, monocytic, erythroleukemia leukemia and myelodysplastic syndrome, chronic leukemia such as but not limited to, chronic myelocytic leukemia, chronic granulocytic leukemia, chronic lymphocytic leukemia, and hairy cell leukemia.

In one embodiment the tumour is, the tumour cells are or the cancer is a lymphoma such as but not limited to Hodgkin's disease and non-Hodgkin's disease. In one embodiment the tumour is, the tumour cells are from or the cancer comprises a hematopoietic tumor of myeloid lineage such as but not limited to acute and chronic myelogenous leukemia, smoldering multiple myeloma, nonsecretory myeloma and osteosclerotic myeloma. In one embodiment the tumour is, the tumour cells are from or the cancer comprises a hematopoietic tumor of lymphoid lineage, including leukemia, acute and chronic lymphocytic leukemia, acute and chronic lymphoblastic leukemia, B-cell lymphoma, T-cell lymphoma, Burkitts lymphoma. In one embodiment the tumour is, the tumour cells are from or the cancer comprises a hematopoietic tumor of B lymphoid lineage. In one embodiment the tumour is, the tumour cells are from or the cancer comprises a hematopoietic tumor of T lymphoid lineage. Additional cancers and related disorders that may be treated or prevented by methods and compositions of the present invention include but are not limited to the following: leukemias; lymphomas; Multiple myelomas; Waldenström's macroglobulinemia; monoclonal gammopathy of undetermined significance; benign monoclonal gammopathy; heavy chain disease; bone and connective tissue sarcomas; brain tumors; breast cancer; adrenal cancer; thyroid cancer; Pancreatic cancer; pituitary cancers; eye cancers; vaginal cancers; vulvar cancer; cervical cancers; uterine cancers; ovarian cancers; esophageal cancers; stomach cancers; colon cancers; rectal cancers; liver cancers; gallbladder cancers; cholangiocarcinomas; lung cancers; testicular cancers; prostate cancers; penal cancers; oral cancers; basal cancers; salivary gland; pharynx cancers; skin cancers; kidney cancers; Wilms' tumor; and bladder cancers. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America.

### 12. Skin Cancer Treatment or Prevention

A further embodiment of the present invention is a method of treating or preventing skin cancer comprising the step of applying lactoferrin and at least one anti-tumour food factor in or on the skin, and/or in the vicinity of the tumor.

In a preferred embodiment the skin is predisposed to skin cancer due to sun exposure. In a preferred embodiment the cancer is a basal cell carcinoma, a squamous cell carcinoma, or a melanoma. Preferably, the ion lactoferrin composition is administered topically, either alone or in combination with standard anti-cancer regimens. Administration in the vicinity of the tumor includes administration near or adjacent to the margins of the tumor or directly in the margin area of the tumor. It is envisioned that lactoferrin inhibits carcinogenesis, stimulates anti-tumour immunity in the local tissue, inhibits tumour angiogenesis, and/or is directly tumouricidal (able to inhibit tumour growth). Briefly, lactoferrin and at least one anti-tumour food factor in a suitable carrier at strengths of 0.1%, 1%, 5%, or 10% is applied twice a day to at-risk skin or cancerous skin lesion. Size progression of the tumour is monitored through CT scans and tumor markers where available.

Doses and treatment regimes can be informed by undertaking preclinical trials in a suitable animal model of skin cancer. A region of the skin of mice is shaved and treated with topical application of a carcinogen (for example, 7,12-dimethylbenz(a)-anthracene (DMBA)) that may be followed by irradiation with UV-B (Bestak, et al., 1996). Lactoferrin may be applied two days after carcinogen treatment or once a cancerous lesion has formed, preferably in the presence of a dermal penetration enhancer (such as 70% laureth sulphate and 30% phenylpiperazine) that could increase skin permeability. Lactoferrin is applied twice a day, or as otherwise required, to the skin or cancerous lesion and tumour growth monitored over a period of weeks to months.

Where appropriate, these methods may be combined with treatments employing any one or more of the anti-tumour agents (including chemotherapeutic agents or immunotherapeutic agents) or anti-tumour therapies described above.

### 13. Lactoferrin Compositions

The lactoferrin and at least one anti-tumour food factor combinations useful herein may be formulated for administration in any chosen dosage form; for example, as food, drink, food additive, drink additive, dietary supplement, nutritional product, medical food, nutraceutical, medicament or pharmaceutical. In one embodiment the present invention relates to use of lactoferrin and at least one anti-tumour food factor in the manufacture of a food, drink, food additive, drink additive, dietary supplement, nutritional product, medical food, nutraceutical, medicament or pharmaceutical. Preferably the composition is formulated for oral or topical administration. Preferably the composition is formulated for oral or parenteral administration. Preferably the composition is for inhibiting tumour growth, inducing apoptosis, inducing apoptosis of tumour cells, treating or preventing cancer, increasing the responsiveness of a subject or the sensitivity of a tumour to a therapy, maintaining or improving one or both of the white blood cell count and red blood cell count of a subject, increasing the production of Th1 and Th2 cytokines within the intestine or a tumour of a subject, or other uses, as described above. Preferably the lactoferrin is as described above. Preferably the anti-tumour factor is one described above.

In one embodiment the lactoferrin and the at least one anti-tumour food factor are formulated for administration separately, simultaneously or sequentially with at least one anti-tumour agent or anti-tumour therapy described above. In one embodiment the lactoferrin and the at least one anti-tumour food factor are formulated for coadministration with the at least one anti-tumour agent or anti-tumour therapy described above. In one embodiment the lactoferrin and the at least one anti-tumour food factor are formulated for sequential administration with the at least one anti-tumour agent or anti-tumour therapy described above. In one embodiment the lactoferrin is included as or is delivered as an adjuvant for the anti-tumour agent or anti-tumour therapy in that the lactoferrin enhances or potentiates the effects of the anti-tumour agent or anti-tumour therapy. At least one anti-tumour food factor may be delivered separately.

In general, for oral administration a dietary (a food, food additive or food supplement for example), nutraceutical or pharmaceutical composition useful herein may be formulated by a skilled worker according to known formulation techniques. For example, foods, food additives or food supplements comprising lactoferrin for use according to the invention include any edible consumer product which is able to carry protein. Examples of suitable edible consumer products include confectionary products, reconstituted fruit products, snack bars, muesli bars, spreads, dips, diary products including yoghurts and cheeses, drinks including dairy and non-dairy based drinks, milk powders, sports supplements including dairy and non-dairy based sports supplements, food and drink additives such as protein sprinkles and dietary supplement products including daily supplement tablets. Suitable nutraceutical compositions useful herein may be provided in similar forms.

In one embodiment a composition of the invention is a milk fraction, preferably a milk protein fraction. In one embodiment the milk fraction comprises at least about 1, 5, 10, 15, 20, 25, 30, 35, 40, 45, 50, 55, 60, 65, 70, 75, 80, 85, 90, 95 or 99% by weight lactoferrin, and useful ranges may be selected from any of these values (for example, from about 1 to about 99% by weight, from about 5 to about 99% by weight, from about 10 to about 99% by weight, from about 15 to about 99% by weight, from about 20 to about 99% by weight, from about 25 to about 99% by weight, from about 30 to about 99% by weight, from about 35 to about 99% by weight, from about 40 to about 99% by weight, from about 45 to about 99% by weight, from about 50 to about 99% by weight, from about 55 to about 99% by weight, from about 60 to about 99% by weight, from about 65 to about 99% by weight, from about 70 to about 99% by weight, from about 75 to about 99% by weight, from about 80 to about 99% by weight, from about 85 to about 99% by weight, from about 90 to about 99% by weight, or from about 95 to about 99% by weight).

A suitable pharmaceutical composition may be formulated with appropriate pharmaceutically acceptable carriers (including excipients and diluents) selected with regard to the intended dosage form and standard pharmaceutical formulation practice. See for example, Remington's Pharmaceutical Sciences, 16th edition, Osol, A. Ed., Mack Publishing Co., 1980.

While the preferred route of administration is oral, it should be understood that any mode of administration may be suitable for any composition of the invention, including administration by multiple routes, including different routes for different agents. Therefore, inhalation (nasal or buccal inhalation) and vaginal and rectal administration of any composition of the invention is also contemplated. Intramedullar, epidural, intra-articular, and intra-pleural administration of any composition of the invention is also contemplated. Administration of lactoferrin or an anti-tumour food factor by a first administration route accompanied by separate, simultaneous or sequential administration of the other agent by a second administration route is also contemplated; for example, oral administration of lactoferrin accompanied by topical administration of the anti-tumour food factor.

A dosage form useful herein may be administered orally as a powder, liquid, tablet or capsule. Suitable dosage forms may contain additional agents as required, including emulsifying, antioxidant, flavouring or colouring agents, or have an enteric coating. Suitable enteric coatings are known. Enteric coatings surrounding the active ingredients and prevent the release of the active ingredients in the stomach but allow release after the dosage form has left the stomach. Dosage forms useful herein may be adapted for immediate, delayed, modified, sustained, pulsed or controlled release of the active components.

Injectable dosage forms may be formulated as liquid solutions or suspensions. Solid forms suitable for solution in, or suspension in, liquid prior to injection may also be prepared. The dosage form may also be emulsified. Lactoferrin and at least one anti-tumour food factor may be mixed with carriers such as, for example, water, saline, dextrose, glycerol, ethanol, or the like and combinations thereof.

Sustained-release preparations may be prepared incorporating lactoferrin and at least one anti-tumour food factor. Suitable examples of sustained-release preparations include semi-permeable matrices of solid hydrophobic polymers containing lactoferrin or a functional variant or functional fragment thereof. The matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides (see U.S. Pat. No. 3,773,919), copolymers of L-glutamic acid and ethyl-L-glutamate, non-degradable ethylene-vinyl acetate, and degradable lactic acid-glycolic acid copolymers such as the LUPRON DEPOT™ (injectable microspheres composed of lactic acid-glycolic acid copolymer and leuprolide acetate).

Topical formulations comprising lactoferrin and at least one anti-tumour food factor may be prepared as lotions, creams, ointments or salves using known carriers for such applications.

In one embodiment a method of the invention comprises administration of a mixture of lactoferrin and at least one functional variant or functional fragment thereof and at least one anti-tumour food factor. Therefore in one embodiment a composition comprises a mixture of lactoferrin and at least one functional variant or functional fragment thereof and at least one anti-tumour food factor. In alternative embodiment a composition comprises a mixture of functional fragments and at least one anti-tumour food factor.

A preferred lactoferrin composition for use herein comprises lactoferrin, or at least one functional variant or functional fragment thereof, or a mixture of lactoferrin and at least one functional variant or functional fragment thereof. Preferably the lactoferrin is bovine lactoferrin or human lactoferrin. Preferably the composition further comprises a digestible protein such as casein or other protective protein. Preferably the composition comprises about 0.1 to 90 wt lactoferrin and about 10 to 90 wt % casein or other protective protein. More preferably the composition consists essentially of about 0.5 to 10 wt % lactoferrin and about 10 to 99 wt % casein or other protective protein. Most preferably the composition consists essentially of about 1 wt lactoferrin and about 20 wt % casein or other protective protein.

Lactoferrin or at least one functional variant or functional fragment thereof may also be administered by parenteral routes including but not limited to subcutaneous, intravenous, intraperitoneal, intramuscular and intratumoural administration. Preferably lactoferrin is administered parenterally by injection. The anti-tumour food factor may be administered by a separate route. Those skilled in the art will be able to prepare suitable formulations for parenteral administration without undue experimentation.

In one embodiment the daily dosage range (by any route) of lactoferrin or metal ion (preferably iron) lactoferrin is about 0.001 to about 100 g per day, preferably about 0.1 to about 30 g, about 0.1 to about 40 g, about 0.1 to about 50 g, about 0.1 to about 60 g, about 0.1 to about 70 g or about 0.1 to about 80 g per day for a 70 kg adult, preferably about 1 mg to about 1.5 g/kg/day, preferably about 10 mg to about 1.5 g/kg/day, preferably about 50 mg to about 500 mg/kg/day. In one embodiment the daily dosage range (by any route) of vitamin D or a vitamin D analogue is about 1,000 IU to about 158,000 IU per day for a 70 kg adult, preferably about 7,000 IU to about 158,000 IU per day for a 70 kg adult, preferably about 10 IU/kg/day to about 2,500 IU/kg/clay, preferably about 100 IU/kg/day to about 2.257 IU/kg/day. In one embodiment the daily dosage range (by any route) of soy protein is about 0.001 to about 100 g per day, preferably about 0.1 to about 30 g, about 0.1 to about 40 g, about 0.1 to about 50 g, about 0.1 to about 60 g, about 0.1 to about 70 g or about 0.1 to about 80 g per day for a 70 kg adult, preferably about 1 mg to about 1.5 g/kg/day, preferably about 10 mg to about 1.5 g/kg/day, preferably about 50 mg to about 500 mg/kg/day. Higher doses are preferred for short-term treatment and prevention and lower doses for long-term treatment and prevention. In one embodiment the daily dose of lactoferrin should comprise about 0.001% to 20%, preferably 0.001% to 2% by weight of the daily diet. In one embodiment the daily dose of soy should comprise about 0.001% to 20%, preferably 0.01% to 20%, preferably 0.01% to 15% by weight of the daily diet. In one embodiment the daily dose of any additional anti-tumour food factor should comprise about 0.001% to 20%, preferably 0.01% to 20% by weight of the daily diet. In one embodiment the daily dose of vitamin D or vitamin D analogue should comprise about 0.0001% to 0.005% by weight of the daily diet.

The lactoferrin may be used alone or in combination with one or more other therapeutic agents, including those described above. When used in combination with another therapeutic agent the administration of the two agents may be separate, simultaneous or sequential. Simultaneous administration includes the administration of a single dosage form that comprises both agents and the administration of the two agents in separate dosage forms at substantially the same time. Sequential administration includes the administration of the two agents according to different schedules, preferably so that there is an overlap in the periods during which the two agents are provided. Suitable agents with which the compositions of the invention can be co-administered include chemotherapeutic and immunotherapeutic agents, and other suitable agents known in the art. Such agents are preferably administered parenterally, preferably by intravenous, subcutaneous, intramuscular, intraperitoneal, intramedullar, epidural, intradermal, transdermal (topical), transmucosal, intra-articular, and intrapleural, as well as oral, inhalation, vaginal and rectal administration.

Additionally, it is contemplated that a composition in accordance with the invention may be formulated with additional active ingredients which may be of benefit to a subject in particular instances. For example, therapeutic agents that target the same or different facets of the disease process may be used.

As will be appreciated, the dose of the composition administered, the period of administration, and the general administration regime may differ between subjects depending on such variables as the severity of symptoms of a subject, the type of disorder to be treated, the mode of administration chosen, and the age, sex and/or general health of a subject.

It should also be appreciated that administration may include a single daily dose or administration of a number of discrete divided doses as may be appropriate. It should also be understood that a person of ordinary skill in the art will be able without undue experimentation, having regard to that skill and this disclosure, to determine an effective dosage regime (including daily dose and timing of administration) for a given condition.

The present invention also relates to a parenteral unit dosage form comprising lactoferrin and at least one anti-tumour agent, for use in combination with at least one anti-tumour food factor. Preferably the at least one anti-tumour agent is selected from paclitaxel, doxorubicin, epirubicin, fluorouracil, cyclophosphamide, methotrexate, an expression plasmid encoding the T cell co-stimulator B7-1 and dendritic cell therapy. Alternatively the agent is selected from any of those described above. Preferably the lactoferrin is as described above.

The present invention also relates to a dietary, nutraceutical or oral pharmaceutical composition comprising, consisting essentially of or consisting of lactoferrin and casein or other protective protein in combination with at least one anti-tumour food factor. Preferably the composition consists essentially of about 0.1 to 90 wt % lactoferrin and about 10 to 90 wt % casein or other protective protein. More preferably the composition consists essentially of about 0.5 to 10 wt % lactoferrin and about 10 to 99 wt % casein or other protective protein. Most preferably the composition consists essentially of about 1 wt % lactoferrin and about 20 wt % casein or other protective protein. Preferably the lactoferrin is as described above.

Various aspects of the invention will now be illustrated in non-limiting ways by reference to the following examples.

### EXAMPLES

### Mice and Reagents

Eight to nine week old male and female C57BL/6 mice (University of Auckland, New Zealand) were used. Each diet group contained 5 mice unless otherwise indicated. Mice were kept in an air-conditioned room with controlled humidity, temperature, and 12 h light:dark cycle. The mouse EL-4 T cell thymic lymphoma was purchased from the American Type Culture Collection (Rockville, Md., USA). It was cultured at 37° C. in DMEM medium (Gibco BRL, Grand Island, N.Y., USA), supplemented with 10% foetal calf serum, 50 U/ml penicillin/streptomycin, 2 mM L-glutamine, 1 mM pyruvate. Palitaxel was obtained from Bristol-Meyers Squibb, WA, USA.

### Lactoferrin Preparation

Bovine lactoferrin was prepared from skim milk (Fonterra Co-Operative Group Limited, New Zealand) using the method of Norris et al (Norris, G E et al., 1989). A SP Big Beads ion exchanger was loaded with skim milk and washed with water. The column was eluted with 0-0.5M NaCl solution and the eluate discarded. The column was then eluted with 0.5-1.0M NaCl and the eluate recovered. The recovered eluate was subjected to UF/DF using a 30 kDa membrane to reduce salts and low molecular weight components. The final retentate contained bovine lactoferrin of between 90 and 93% purity. The lactoferrin extract obtained had natural levels of iron-saturation of approximately 15% and is referred to “natural bLf” in the following Examples. Iron-saturated bovine lactoferrin extract (100% saturated) was prepared from natural bLf by the method of Law et al (1997).

### Lactoferrin Treatment

The experimental diets were prepared by Crop & Food Research, Palmerston North, New Zealand using as a base the powdered AIN93G formulation. Casein was used as the protein source in the AIN93G diet, and contained no lactoferrin. It was supplemented in the experimental diets with natural bLf or iron-saturated bovine lactoferrin prepared as described above, such that the total protein content of the diet was unchanged. The diet contained 28 g of iron-saturated bovine lactoferrin or 28 g of natural bLf extract per 2400 g of diet. Fresh diet was provided biweekly, and mice had free access to food and water throughout the study.

### Experimental Tumor Model and Therapies

Tumors were established by s.c. injection of 2×10⁵ EL-4 cells into the left flank of mice, and growth determined by measuring two perpendicular diameters. Animals were euthanized when tumors reached more than 1.0 cm in diameter, in accord with Animal Ethics Approval (University of Auckland). All experiments included 5 mice per treatment group, unless otherwise indicated. Paclitaxel (30 mg dissolved in 5 ml of Cremophor® EL and dehydrated alcohol) was diluted in 0.9% NaCl and administered i.p. at 30 mg/Kg. Tumors were injected with 180 μl of DNA (180 μg)/liposome complexes, as described previously (Kanwar, et al., 1999 and Kanwar, et al., 2000).

### Measurement of the Generation of Antitumor Cytotoxic T-lymphocytes (CTLs)

Splenocytes were harvested 28 days following tumour cell injection, as specified. They were incubated at 37° C. with EL-4 target cells in graded E:T ratios in 96-well round-bottom plates. After a 4-hour incubation, 50 μl of supernatant was collected, and lysis was measured using the Cyto Tox 96 Assay Kit (Promega, Madison, Wis., USA). Background controls for non-specific target and effector cell lysis were included. After background subtraction, percentage of cell lysis was calculated using the formula: 100×(experimental-spontaneous effector-spontaneous target/maximum target-spontaneous target).

### Measurement of Apoptosis

For in situ detection of apoptotic cells, tumors were excised and immediately frozen in dry ice, and stored at −70° C. Frozen serial sections of 6-μm thickness were fixed with paraformaldehyde solution (4% in PBS, pH 7.4), and permeabilized with a solution containing 0.1% Triton X-100 and 0.1% sodium citrate. They were incubated with 20 μl TUNEL reagent (In Situ apoptosis detection kit from Boehringer Mannheim, Germany) for 60 min at 37° C., and examined by fluorescence microscopy. Adjacent sections were counter-stained with haematoxylin to count the total number of cells, or the number of apoptotic cells in ten randomly selected fields (magnification of ×40). The apototic index (AI) was calculated as the number of apoptotic cells×100/total number of nucleated cells. For detection of apoptotic cells in vitro, the numbers of apoptotic and necrotic tumour cells were measured by staining with annexin-V-fluos, TUNEL, and trypan blue, as described previously (Kanwar, et al., 2001).

### Assessment of Vascularity

To determine tumor vascularity, 10-μm frozen tumor sections were immunostained with the anti-CD31 antibody MEC13.3, and an anti-CD105 mAb. Stained blood vessels were counted from five mice in six blindly chosen random fields (0.155 mm2) at ×40 magnification. To visualize blood vessels open to flow, DiO7 (Molecular Probes, Eugene, Oreg.) was injected into the tail vein at a concentration of 1.0 mg/Kg one minute prior to collecting tissues.

### Statistical Analysis

Results were expressed as mean values±standard deviation (S.D.), and a Student's t test was used for evaluating statistical significance. A value of p<0.05 denotes statistical significance, whereas p<0.001 denotes results that are highly significant.

### Example 1

Bovine lactoferrin of greater than 90% purity was sourced from the Fonterra Co-operative Group. For the preparation of apo-Lf, a solution of Lf at approximately 80 mg/mL in milliQ water (pH˜5.7) was adjusted to pH 2.08 by careful addition of 6 M HCl. The solution was stirred at RT for 1 h then dialysed against 10 volumes of 0.1 M citric acid overnight at 4° C. using SpectraPor tubing with a nominal molecular weight cut-off of 3.5 kDa (Spectrum Companies, Ranco Dominguez, Calif., USA). The dialysis fluid was changed twice over a 24 h period, and the Lf solution freeze-dried to a white semi-crystalline powder. For preparation of 50% Fe-saturated lactoferrin, an 8% solution of lactoferrin in 0.1 M sodium bicarbonate was adjusted to pH 8.2 with careful addition of 6 M NaOH. An appropriate volume of 50 mM ferric nitrilo-triacetate (Fe-NTA) (Bates et al., 1967; Brock & Arzabe, 1976) was added to give ˜50% saturation of the lactoferrin (allowing for the purity of the Lf and its native Fe saturation of ˜12%). After stirring for 1 h at RT, the solution (pH 8.01) was dialysed against 10 volumes of milli-Q water overnight at 4° C. using SpectraPor tubing as above. The dialysis fluid was changed twice over a 24 h period and the Lf solution freeze-dried to a salmon red semi-crystalline powder. Lactoferrin of ˜100% Fe saturation was prepared essentially as for the 50% Fe-saturated material except that the amount of Fe-NTA was adjusted accordingly, and following addition of Fe-NTA, the pH was re-adjusted to 8.0 with careful addition of 6 M NaOH. The final product was a deep salmon red semi-crystalline powder. Fe saturation levels of the final products were verified by spectrophotometric titration (Bates et al., 1967; Brock & Arzabe, 1976). The apo-lactoferrin was approximately 5% Fe-saturated.

A single native lactoferrin preparation was used to generate three additional preparations of lactoferrin, each containing different levels of Fe-saturation. The Fe was removed by citric acid chelation to provide apoLf (5% Fe-saturated), or alternatively lactoferrin was supplemented with Fe to 50% and 100% saturation. Fully Fe-saturated Lf, 50% Fe-saturated Lf, native Lf, and apoLf were fed orally to mice to compare their anti-tumour activities. EL-4 tumour cells (2×10⁵) were injected into the left flank of C57BL/6 mice following two weeks on lactoferrin diets containing 20 g of Lf per 2.4 Kg, or on the control diet. In this particular experiment, the level of Fe-saturation did not appear to effect the growth rate of tumours, except for mice fed the Fe-saturated diet where one of ten mice completely rejected the tumour challenge (FIG. 1A). Paclitaxel (30 mg/Kg) was injected i.p. once tumours reached approximately 0.6 cm in diameter. As before EL-4 tumours of this size were completely resistant to paclitaxel treatment in mice fed the control diet (FIG. 1A). In contrast, the tumours of mice maintained on an iron-saturated bovine Lf-supplemented diet regressed to less than half their size within two weeks of administering paclitaxel, and disappeared altogether a week later (FIG. 1A). The other three preparations of lactoferrin containing lesser levels of Fe were not able to synergize with paclitaxel to eradicate tumours but were still effective to make tumours sensitive to paclitaxel so that tumours were reduced in size. Their efficacy correlated with the degree of Fe-saturation, such that the efficacy of 50% Fe-saturated Lf>native Lf>apoLf. In summary, Fe-saturated Lf, but not lesser Fe-saturated forms of bovine Lf, was able to change a tumour that was completely resistant to chemotherapy into a tumour that was exquisitely sensitive to chemotherapy.

Splenocytes were harvested from the mice described in FIG. 1A at day 77 (or day 56 in the case of controls) and tested for their cytolytic activity against EL-4 target cells. The anti-tumour cytolytic activity of splenocytes obtained from the one mouse fed Fe-saturated lactoferrin which completely resisted the tumour challenge was significantly (P<0.001) increased (by 6-fold), compared to control mice (FIG. 1B). The anti-tumour cytolytic activity of splenocytes was significantly increased in the remaining nine animals treated with fully Fe-saturated Lf (by 6.5-fold, <0.001), and to a lesser extent in mice fed 50% Fe-saturated Lf (by 1.5-fold, (P<0.001), native Lf (by 3.4-fold, (P<0.001), and apoLf (by 2.4-fold, (P<0.001) in combination with paclitaxel treatment. Thus, fully Fe-saturated Lf has the greatest effect in stimulating anti-tumour cytolytic activity in combination with chemotherapy, in accord with the ability of the latter treatment to completely eradicate tumours. Referring to FIG. 1B, the percent cytotoxicity is plotted against various effector-to-target cell ratios (E:T ratios); each point represents the mean percent cytotoxicity obtained from 5 mice; and the bar represents 95% confidence intervals.

### Example 2

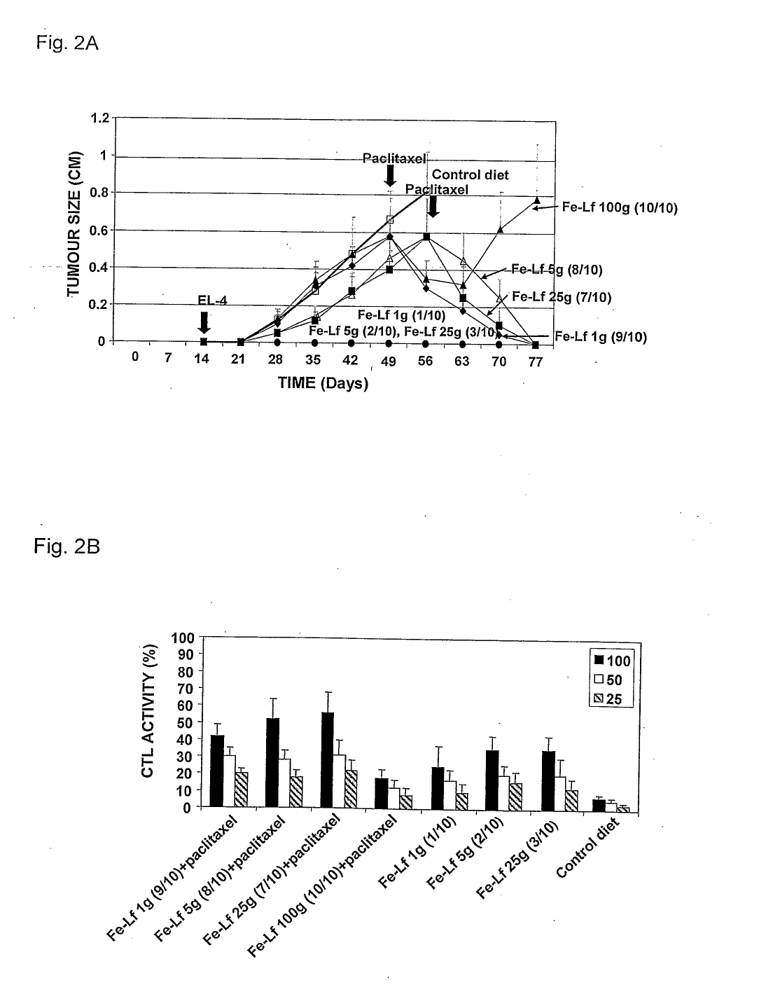

Mice were fed the control diet, and the same diet supplemented with different levels of 100% Fe-saturated Lf ranging from 0, 1, 5, 25, and 100 g per 2.4 Kg of diet. EL-4 tumour cells (2×10⁵) were injected into the left flanks of C57BL/6 mice following two weeks on the Lf diets, or control diet. The tumour growth rate of mice fed the lowest and highest doses of Fe-saturated Lf did not differ greatly from that of mice fed the control diet, whereas in contrast, tumours in mice fed diets containing 5 and 25 g of Fe-saturated Lf per 2.4 Kg of diet grew significantly (p<0.05 at days 35-49) more slowly compared to tumours of mice fed the control diet (FIG. 2A). In this particular experiment, one of ten mice fed the 1 g'Fe-Lf diet, two of ten mice fed the 5 g Fe-Lf diet, and three of ten mice fed the 25 g Fe-Lf diet completely rejected the tumour challenge. Paclitaxel (30 mg/Kg) was injected i.p. once turnouts reached approximately 0.6 cm in diameter. The tumours of mice fed all but the 100 g Fe-Lf diet rapidly regressed and completely disappeared over the following three to four weeks. In contrast, tumours in mice fed the highest dose of Fe-saturated Lf regressed over two weeks, but then re-grew. It was concluded that a diet containing approximately 5 to 25 g of Fe-saturated Lf per 2.4 Kg of diet had the greatest efficacy in inhibiting tumour growth, and rendering tumours susceptible to chemotherapy.

Splenocytes were harvested from the mice described in FIG. 2A at day 77 (or day 56 in the case of controls) and tested for their cytolytic activity against EL-4 target cells. The anti-tumour cytolytic activities of splenocytes obtained from the 6 of 30 mice that rejected the tumour challenge after being fed the 1, 5, and 25 g Fe-saturated Lf diets were significantly increased (by 2.6 to 4-fold, p<0.001) compared to controls (FIG. 2B). The increase in anti-tumour cytolytic activity of splenocytes after injection of tumours with paclitaxel was greatest for mice fed the 5 (6.7-fold, p<0.001) and 25 g (7-fold, p<0.001) Fe-saturated Lf diets, in accord with the ability of the latter treatments to cause rapid and complete tumour regression. In contrast, the increase in anti-tumour cytolytic activity was lowest for mice fed the 100 g Fe-saturated Lf diet (1.5-fold, p<0.001), which did not synergize with paclitaxel to eradicate tumours, although this dose still rendered the tumour susceptible to one dose of paclitaxel. Referring to FIG. 2B, the percent cytotoxicity is plotted against various effector-to-target cell ratios (E:T ratios); each point represents the mean percent cytotoxicity obtained from 5 mice; and the bar represents 95% confidence intervals.

### Example 3

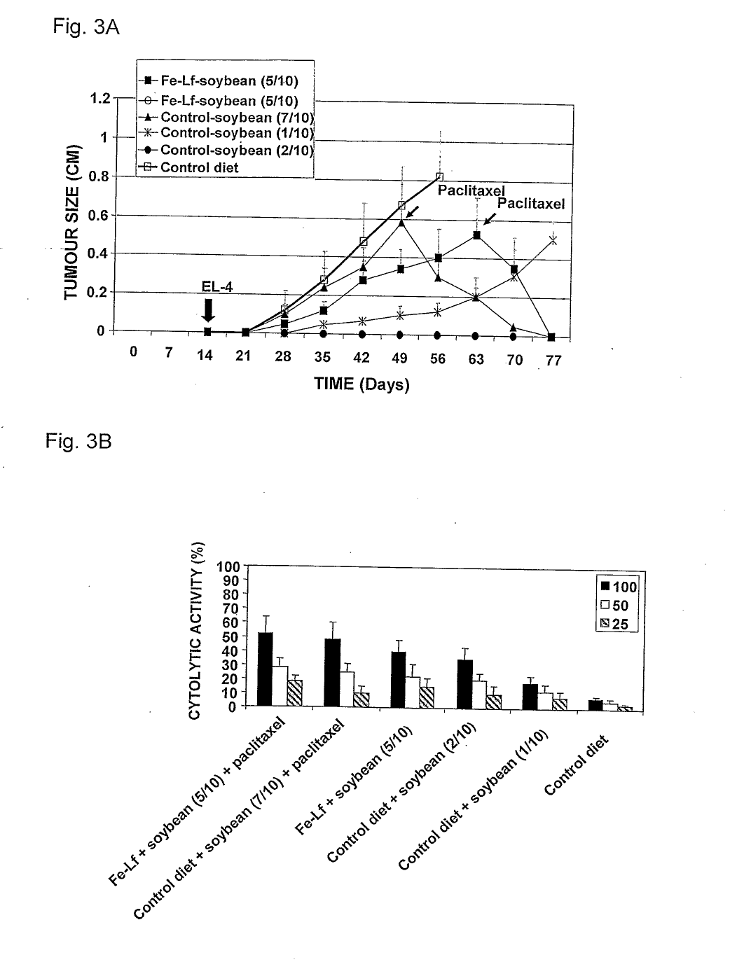

Mice were fed either a casein-based control diet, a soy protein-based control diet, or a soy protein-based diet containing 28 g of 100% Fe-saturated Lf per 2.4 Kg of diet, and after two weeks on the diets EL-4 tumour cells (2×10⁵) were injected into the left flank of all mice. Perfect soy protein obtained from Aussie Bodies comprises Supro® protein, a water-washed soy protein isolate. The soy protein diet had no significant (p>0.05 at day 49) impact on tumorigenesis in the majority (7 out of 10) of mice, but nevertheless, it significantly (p<0.001) inhibited tumorigenesis in one mouse, and completely prevented tumorigenesis in two other mice (FIG. 3A). The efficacy of Fe-saturated Lf appeared to be enhanced on a soy protein background, since five of ten mice completely rejected the tumour challenge, and tumour growth was significantly reduced (p<0.001 at day 63) in the five mice which developed tumours.

Paclitaxel (30 mg/Kg) was injected i.p. into mice which developed tumours once tumours had reached 0.5 to 0.6 cm in diameter. The tumours of the seven tumour-bearing mice fed soy protein immediately regressed and continued to regress slowly until they disappeared altogether four weeks later. The turnouts of mice fed Fe-saturated Lf on a base diet of soy protein also regressed, but took only two weeks to completely disappear. In summary, whereas Fe-saturated Lf and soy protein both weakly inhibit tumorigenesis, together they are more effective in that they synergize to completely prevent the development of tumours in half the mice. Soy protein, like Fe-saturated Lf, renders tumours susceptible to chemotherapy, but is not quite as effective as Fe-saturated Lf. In combination, they were slightly more effective than Fe-saturated Lf in increasing the susceptibility of turnouts to chemotherapy.

Splenocytes were harvested from the mice described in FIG. 3A at day 77 (or day 56 in the case of controls) and tested for their cytolytic activity against EL-4 target cells. The anti-tumour cytolytic activity of splenocytes obtained from the two mice fed soy protein, which completely resisted the tumour challenge was significantly (P<0.001) increased (by 3.9-fold), compared to mice fed the control diet (FIG. 3B), whereas the cytolytic activity of the one mouse which strongly resisted the tumour challenge was increased to a lesser degree (1.6-fold, P<0.001). The anti-tumour cytolytic activity of splenocytes was increased further in the five of ten mice (by 4.6-fold, P<0.001) fed the Combination of Fe-saturated Lf and soy protein that completely resisted the tumour challenge. There was a further increase in anti-tumour cytolytic activity with paclitaxel treatment for mice fed either soy protein (by 5.8-fold, P<0.001), or the combination of Fe-saturated Lf and soy protein (by 6.3-fold, P<0.001), compared to mice fed the control diet. Referring to FIG. 3B, the percent cytotoxicity is plotted against various effector-to-target cell ratios (E:T ratios); each point represents the mean percent cytotoxicity obtained from 5 mice; and the bar represents 95% confidence intervals.

Groups of 5 mice were fed either a casein-based control diet, or diets containing 5 g of 100% Fe-saturated Lf per 2.4 Kg of diet supplemented with 5, 20, 80, or 320 g of soy protein (Aussie Bodies Supro® protein) per 2.4 Kg of diet. After two weeks on the diets EL-4 tumour cells (2×10⁵) were injected into the left flank of all mice. Tumours of control mice reached 0.8 cm in diameter six weeks after injecting the tumour cells, whereas none of the twenty mice fed the combination of Fe-saturated Lf and soy protein developed a single tumour (data not shown). Splenocytes were harvested from the above mice six weeks after injecting the tumour cells in the case of controls, and 10 weeks in the case of the treated mice. The splenocytes were tested for their cytolytic activity against EL-4 target cells. The anti-tumour cytolytic activity of splenocytes obtained from mice fed the various combinations of Fe-saturated Lf and soy protein was increased 6.1 to 8.3-fold, respectively, compared to that of control mice (data not shown).

### Example 4

The anti-tumour activity of diets containing Fe-saturated Lf and increasing amounts of vitamin D3 (cholecalciferol) were examined. Mouse diets supplemented with 28 g Fe-saturated Lf per 2.4 Kg of diet were further supplemented with feed grade vitamin D3 (cholecalciferol, 500,000 IU/g) in escalating doses of 2 mg (1000 IU), 9.2 mg (4,600 IU), 21.2 mg (10,600), and 45.2 mg (22,600 IU)/Kg of diet. Since mice eat ˜3 g of diet per day this equates to consumption of approximately 3, 13.8, 31.8 and 67.8 IU/day. The equivalent human dose on a weight to weight basis would be approximately 7,000, 32,000, 74,000, and 158,000 IU; and on a body surface area (m²) would be 732, 3,370, 7,769, and 16,564 IU.

Mice were fed the control AIN-93 diet which contains 2 mg (1000 IU)/Kg of vitamin D or the three diets containing larger amounts of vitamin D.

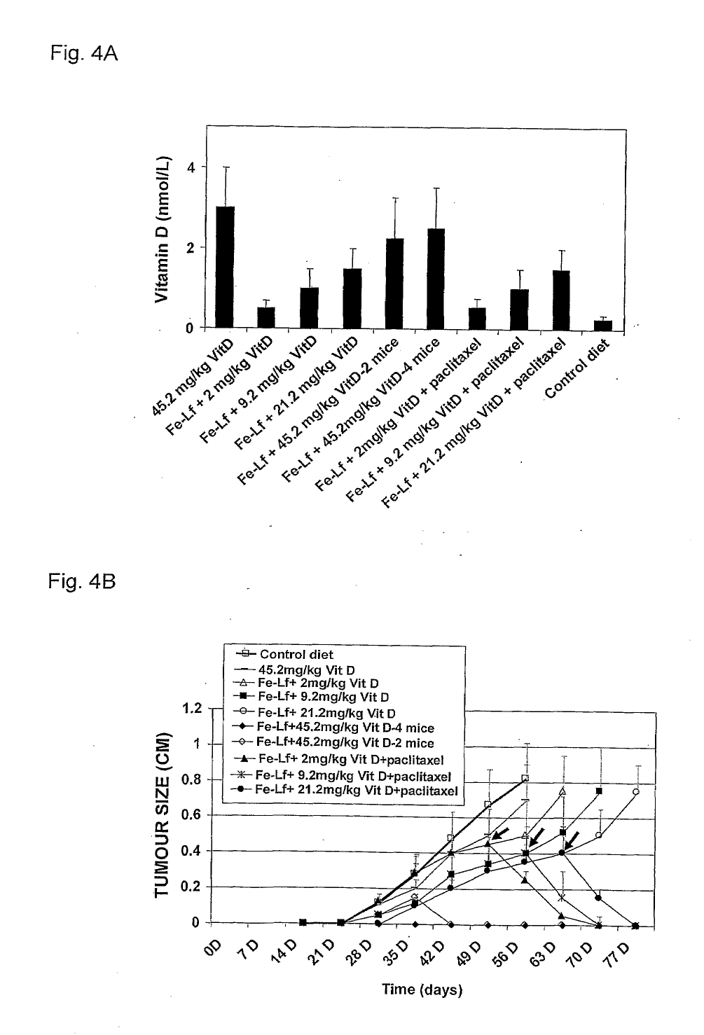

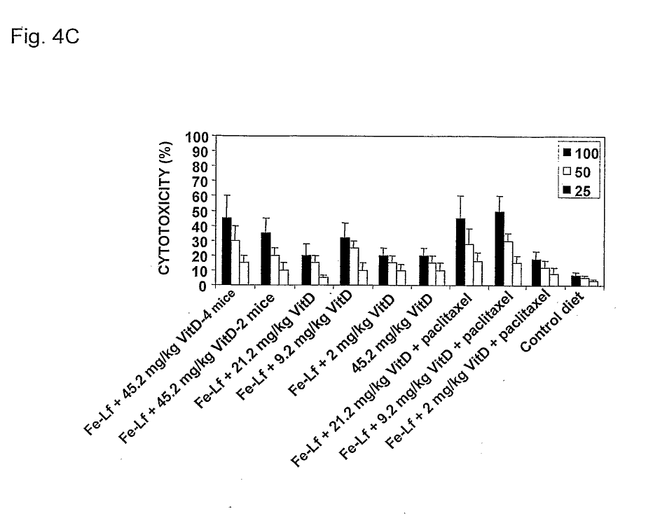

Serum samples were analyzed for 1,25(OH)2D3 content using an enzyme immunoassay kit from Immunodiagnostic Systems Ltd (IDS Ltd), Tyne & Wear, UK. The serum levels of 1,25(OH)2D3, the active form of vitamin D3, increased with increasing levels of cholecalciferol in the diet. Feeding the Lf-containing diet having the lowest amount of cholecalciferol (2 mg/Kg; identical to that for the base AIN-93 diet) resulted in a serum concentration of 0.5 nM 1,25(OH)2D3, whereas the Lf-containing diet having the highest amount (45.2 mg/Kg) of cholecalciferol resulted in an 6-fold higher concentration of 3 nM 1,25(OH)2D3 (FIG. 4A). The serum levels of 1,25(OH)2D3 remained relatively constant irrespective of treatment with paclitaxel. In comparison, 1,25(OH)2D3 in rat serum has been recorded at 0.08 nM.

EL-4 tumour cells (2×10⁵) were injected into the left flanks of C57BL/6 mice following two weeks on the Lf+vitamin D diets, or control diet. Diets in which Fe-saturated Lf was combined with 2, 9.2, and 21.2 mg/Kg of vitamin D inhibited tumour growth (at day 56) by 39 (p<0.05), 51 (p<0.001), 53% (p<0.01), respectively, compared to the control diet (FIG. 4B). In contrast, tumours did not develop in four of six mice fed the highest level of vitamin D (45.2 mg/Kg of diet) in combination with Fe-saturated Lf, and small (0.15 cm diameter) tumours developed by day 28 in two of six mice, but then promptly regressed on day 35 and completely disappeared. In summary, the combination of Fe-saturated Lf and a relatively high dose vitamin D (roughly equivalent to 16,564 to 158,200 IU/day in humans) is able to completely prevent the development of tumours in mice. The efficacy of a Fe-saturated Lf-based therapy is not significantly enhanced until the level of vitamin. D is increased at least by an order of magnitude. Paclitaxel (30 mg/Kg) was injected i.p. into mice which developed tumours once tumours had reached ˜0.4 cm in diameter. The tumours that developed in mice fed Fe-saturated Lf in combination with the lesser amounts of vitamin D were completely eradicated by paclitaxel, indicating they remained exquisitely sensitive to this drug.

Splenocytes were harvested from the mice described in FIG. 4B at day 77 (or day 56 in the case of controls) and tested for their cytolytic activity against EL-4 target cells. The anti-tumour cytolytic activity of splenocytes obtained from the mice fed Fe-saturated Lf in combination with the highest dose of cholecalciferol (42.2 mg/Kg), which completely resisted the tumour challenge, was significantly increased (by 5.4-fold, p<0.001) compared to mice fed the control diet, and by 1.9 to 3.6-fold, compared to mice fed Lf in combination with the lesser amounts of cholecalciferol (FIG. 4C). High dose vitamin D by itself increased anti-tumour cytolytic activity to the same extent (by 1.9-fold) as Fe-saturated Lf.

### INDUSTRIAL APPLICATION

The methods, medicinal uses and compositions of the present invention have utility in inhibiting tumour growth, maintaining or improving one or both of the white blood cell count and red blood cell count, stimulating the immune system and in treating or preventing cancer. The methods and medicinal uses may be carried out by employing dietary (as foods or food supplements), nutraceutical or pharmaceutical compositions. Those persons skilled in the art will understand that the above description is provided by way of illustration only and that the invention is not limited thereto.

### REFERENCES

Ainscough, E W, Brodie A, M.; Plowman J E. The chromium, manganese, cobalt and copper complexes of human lactoferrin. Inorganica Chimica Acta 1979, 33 (2) 149-53.
Baker E N, Baker H M, Kidd R D., Lactoferrin and transferrin: functional variations on a common structural framework. Biochem Cell Biol. 2002; 80(1):27-34.
Banni S, Angioni E, Murru E, Carta G, Melis M P, Bauman D, Dong Y, Ip C. Vaccenic acid feeding increases tissue levels of conjugated linoleic acid and suppresses development of premalignant lesions in rat mammary gland. Nutr Cancer. 2001; 41:91-7.
Bates G W, Billups C, Saltman P, (1967). The kinetics and mechanism of iron exchange between chelates and transferrin. 1. The complexes of citrate and nitrilotriacetic acid. J. Biol. Chem 242, 2810-2815.
Bates G W and Schlabach M R (1973). The reaction of Ferric salts with Transferrin. J. Biol. Chem 248, 3228-3232.
Beer T M, Munar M, Henner W D. A Phase I trial of pulse calcitriol in patients with refractory malignancies: pulse dosing permits substantial dose escalation. Cancer. 2001; 91:2431-9.
Beer T M, Eilers K M, Garzotto M, Egorin M J, Lowe B A, Henner W D. Weekly high-dose calcitriol and docetaxel in metastatic androgen independent prostate cancer. J Clin Oncol. 2003; 21:123-8.
Beer T M, Myrthue A., Calcitriol in cancer treatment: from the lab to the clinic. Mol Cancer Ther. 2004; 3:373-81.
Belobrajdic D P, McIntosh G H. Dietary butyrate inhibits NMU-induced mammary cancer in rats. Nutr Cancer. 2000; 36:217-23.
Bestak R, Halliday G M. Sunscreens protect from UV-promoted squamous cell carcinoma in mice chronically irradiated with doses of UV radiation insufficient to cause edema. Photochem Photobiol. 64:188-93, 1996.
Bowie J U, Reidhaar-Olson J F, Lim W A, Sauer R T. Deciphering the message in protein sequences: tolerance to amino acid substitutions. Science. (1990) 247(4948):1306-10.
Brock J H, Arzabe F R (1976), Cleavage of diferric bovine transferrin into two monoferric fragments. FEBS Lett 69, 63-66.
Brock J H. The physiology of lactoferrin. Biochem Cell Biol 2002; 80:1-6.
Cadwell, R. C. and G. F. Joyce. Randomization of genes by PCR mutagenesis. PCR Methods Appl. 1992; 2: 28-33.
Colston K W, Pirianov G, Bramm E, Hamberg K J, Binderup L. Effects of Seocalcitol (EB1089) on nitrosomethyl urea-induced rat mammary tumors. Breast Cancer Res Treat. 2003; 80:303-11.
Conneely O M. Antiinflammatory activities of lactoferrin. J Am College Nutr 2001; 20:389 S-395S.
Corl B A, Barbano D M, Bauman D E, Ip C. cis-9, trans-11 CLA derived endogenously from trans-11 18:1 reduces cancer risk in rats. J Nutr. 2003; 133:2893-900.
Dass C R. Tumour angiogenesis, vascular biology and enhanced drug delivery. J Drug Target. 2004 June; 12(5):245-55
Facon M J, Skura B J. Antibacterial activity of lactoferricin, lysozyme and EDTA against Salmonella enteritidis. Int. Dairy J. 1996, 6 (3) 303-13.
Foley A A, Bates G W. The purification of lactoferrin from human whey to batch extraction Analytica Biochemistry 1987, 162 (1) 296-300.
Freireich E J, Gehan E A, Rall D P, Schmidt L H, Skipper H E., Quantitative comparison of toxicity of anticancer agents in mouse, rat, hamster, dog, monkey, and man. Cancer Chemother Rep. (1966) 50(4):219-44.
Guyton K Z, Kensler T W, Posner G H. Cancer chemoprevention using natural vitamin D and synthetic analogs. Annu Rev Pharmacol Toxicol. 2001; 41:421-42.
Guyton K Z, Kensler T W, Posner G H. Vitamin D and vitamin D analogs as cancer chemopreventive agents. Nutr Rev. 2003; 61:227-38.
Harris D M, Go V L. Vitamin D and colon carcinogenesis. J. Nutr. 2004; 134:3463 S-3471S.
Holick M F. Vitamin D: importance in the prevention of cancers, type 1 diabetes, heart disease, and osteoporosis. Am J Clin Nutr. 2004; 79:362-71.
Inaba K, Inaba M, Romani N, Aya H, Deguchi M, Ikehara S, Muramatsu S, Steinman R M. Generation of large numbers of dendritic cells from mouse bone marrow cultures supplemented with granulocyte/macrophage colony-stimulating factor. J Exp Med. 1992; 176:1693-1702.
Kanwar J, Berg R, Lehnert K, and Krissansen G W. Taking lessons from dendritic cells: Multiple xenogeneic ligands for leukocyte integrins have the potential to stimulate anti-tumour immunity. Gene Therapy 1999; 6:1835-44.
Kanwar J R, Kanwar R, Pandey S, Ching L-M, and Krissansen G. W. Vascular attack by 5,6-dimethylxanthenone-4-acetic acid combined with B7.1-mediated immunotherapy overcomes immune-resistance and leads to the eradication of large tumors. Cancer Res 2000; 61:1948-56.
Kanwar J R, Shen W P, Kanwar R K, Berg R W, Krissansen G W. Effects of survivin antagonists on growth of established tumors and B7-1 immunogene therapy. J Natl Cancer Inst. 2001; 93:1541-52.
Karthikeyan S, Sujata Sharma, Ashwani K. Sharma, M. Paramasivam, Savita Yadav, A. Srinivasan and Tej P. Singh, Structural variability and functional convergence in lactoferrins, Current Science (1999) 77(2):241
Kawakami H et al. Effect of lactoferrin on iron solubility under neutral conditions. BiosScience Biotech Biochem 1993; 57(8) 1376-1377.
Kerwin S M. Soy saponins and the anticancer effects of soybeans and soy-based foods. Curr Med Chem Anti-Canc Agents. 2004; 4:263-72.
Kimmerlin T, Seebach D. 100 years of peptide synthesis: ligation methods for peptide and protein synthesis with applications to beta-peptide assemblies. J Pept Res. 2005 February; 65(2):229-60.
Kris-Etherton P. Bioactive compounds in foods: Their role in prevention of cardiovascular disease and cancer. American Journal of Medicine 113: 71S-88S, 2002.
Law, B. A. and Reiter, B., The isolation and bacteriostatic properties of lactoferrin from bovine milk whey. J. Dairy Res. (1977) 44:595-599.
Legrand D, Mazurier J, Metz-Boutigue M-H, Jolles J, Jo;;es P, Montreuil J & Spik G (1984). Characterization and localization of an iron-binding 18-kDa glycopeptide isolated from the N-terminal half of human lactotransferrin. Biochimica et Biophysica acta 787, 90-96.
Leung, D. W., Chen, E. Y., Goeddel, D. V. A Method for Random Mutagenesis of a Defined DNA Segment Using a Modified Polymerase Chain Reaction. Technique 1989; 1:11-15.
Masson P L, Heremans J F. Studies on lactoferrin, the iron-binding protein of secretions. Protides of the Biological Fluids 1966, 14, 115-24.
Masso-Welch P A, Zangani D, Ip C, Vaughan M M, Shoemaker S, Ramirez R A, Ip M M. Inhibition of angiogenesis by the cancer chemopreventive agent conjugated linoleic acid. Cancer Res. 2002; 62:4383-9.
Mata L, Castillo H, Sanchez L, Puyol P, Calvo M. Effect of trypsin on bovine lactoferrin and interaction between the fragments under different conditions. J Dairy Res. (1994) 61(3):427-32.
Metz-Boutigue M H, Jolles J, Mazurier J, Schoentgen F, Legrand D, Spik G, Montreuil J, Jolles P. Human lactotransferrin: amino acid sequence and structural comparisons with other transferrins. Eur J. Biochem. 1984; 145(3):659-76.
Moore S A, Anderson B F, Groom C R, Haridas M, Baker E N. Three-dimensional structure of diferric bovine lactoferrin at 2.8 A resolution. J Mol. Biol. 1997 Nov. 28; 274(2):222-36.
Nakagawa K, Sasaki Y, Kato S, Kubodera N, Okano T. 22-Oxa-1alpha,25-dihydroxyvitamin D3 inhibits metastasis and angiogenesis in lung cancer. Carcinogenesis. 2005; 26:1044-54.
Nguyen L T, Schibli D J, Vogel J. Structural studies and model membrane interactions of two peptides derived from bovine lactoferricin. Journal of Peptide Science 2005, 11 (7) 379-89.
Nolan E, Donepudi M, Van Weelden K, Flanagan L, Welsh J E. Dissociation of vitamin D3 and anti-estrogen mediated growth regulation in MCF-7 breast cancer cells. Mol Cell Biochem 1998; 188:13-20.
Norris G E, Baker H M, Baker E N. Preliminary crystallographic studies on human apo-lactoferrin in its native and deglycosylated forms. J Mol. Biol. 1989; 209(2):329-31.
Park E J and Pezzuto J M. Botanicals in cancer prevention. Cancer and Metastasis Reviews 21: 231-255, 2002.
Peleg S, Posner G H. Vitamin D analogs as modulators of vitamin D receptor action. Curr Top Med Chem. 2003; 3:1555-72.
Pierce A, Colavizza D, Benaissa M, Maes P, Tartar A, Montreuil J, Spik G. Molecular cloning and sequence analysis of bovine lactotransferrin. Eur J Biochem. 1991; 196(1):177-84.
Sambrook, J.; Fritsch, E. F.; Maniatis, T. (1989). Molecular Cloning: A Laboratory Manual. Cold Spring Harbour Lab Press, Cold Spring Harbour, New York.
Saul A W. Vitamin D: Deficiency, diversity and dosage. J Orthomolecular Med. 2003; 18:194-204.
Steinman R M, Turley S, Mellman I, Inaba K. The induction of tolerance by dendritic cells that have captured apoptotic cells. J Exp Med. 2000 7; 191:411-416.
Stemmer W P. DNA shuffling by random fragmentation and reassembly: in vitro recombination for molecular evolution. Proc Natl Acad Sci USA 1994; 91:10747-10751.
Sun, X., Kanwar, J. R., Leung, E., Lehnert, K., Wang, D., and Krissansen, G. W. Gene transfer of antisense hypoxia inducible factor-1α enhances the therapeutic efficacy of cancer immunotherapy. Gene Therapy 8: 638-645, 2001.
Superti F, Siciliano R, Rega B, Giansanti F, Valenti P, Antonini G (2001) Involvement of bovine lactoferrin metal saturation, sialic acid and protein fragments in the inhibition of rotavirus infection. Biochim Biophya Acta 1528 (2-3) 107-15.
Tomita M, Bellamy W, Takase M, Yamauchi K, Wakabayashi H, Kawasi K. Potent antibacterial peptides generated by pepsin digestion of bovine lactoferrin. J. Dairy Science 1991, 74 (12) 4137-4
Tomita M, Takase M, Bellamy W, Shimamura S. A review: the active peptide of lactoferrin. Acta Paediatr. Jpn. 1994, 36, 585-91.
Tomita M, Takase, M, Wakabayashi H & Bellamy W (1994) Antimicrobial Peptides of Lactoferrin in Lactoferrin Structure and Function, pp 209-218. Eds T W Hutchens, S V Rumball, B Lonnerdal, Plenum Press, New York.
Tomita M, Wakabayashi H, Yamauchi K, Teraguchi S, Hayasawa H; Bovine lactoferrin and lactoferricin derived from milk: production and applications. Biochemistry and Cell Biology 2002, 80 (1) 109-12.
Tsuda H, Sekine K, Fujita K, Iigo M. Cancer prevention by bovine lactoferrin and underlying mechanisms—a review of experimental and clinical studies. Biochem Cell Biol 2002; 131-36.
Tsuji S, Hirata Y, Matsuoka K. Two apparent molecular forms of bovine lactoferrin. J Dairy Sci. 1989; 72(5):1130-6.
van der Kraan M I A, Groenink J, Nazmi K, Veerman E C I, Bolscher J G M, Nieuw Amerongen A V. Lactoferrampin: a novel antimicrobial peptide in the N1-domain of bovine lactoferrin. Peptides 2004, 25 (2) 177-83.
van Veen H A, Geerts M E, van Berkel P H, Nuijens J H. The role of N-linked glycosylation in the protection of human and bovine lactoferrin against tryptic proteolysis. Eur. J. Biochem. (2004) 271(4): 678-684.
van Weelden K, Flanagan L, Binderup L, Tenniswood M, Welsh J E. Apoptotic regression of MCF-7 xenografts in nude mice treated with the vitamin D analog EB1089. Endocrinology 1998; 139:2102-10.
Viejo-Diaz M, Andrés M T, Pérez-Gil J, Sánchez M, Fierro J F. Potassium Efflux Induced by a New Lactoferrin-Derived Peptide Mimicking the Effect of Native Human Lactoferrin on the Bacterial Cytoplasmic Membrane. Biochemistry (Moscow) 2003, 68 (2) 217-27.
Ward P P, Uribe-Luna S, Conneely O M. Lactoferrin and host defense. Biochem Cell Biol 2002; 80:95-102.
Watkins S M, Carter L C, Mak J, Tsau J, Yamamoto S, German J B. Butyric acid and tributyrin induce apoptosis in human hepatic tumour cells. J Dairy Res. 1999; 66:559-67.
Weinberg E D. Human lactoferrin: a novel therapeutic with broad spectrum potential. Pharm Pharmacol 2001; 53:1303-10.
Yee Y K, Chintalacharuvu S R, Lu J, Nagpal S. Vitamin D receptor modulators for inflammation and cancer. Mini Rev Med Chem. 2005; 5:761-78.
Yoshida, S, and Xiuyn, Ye. Isolation of Lactoperoxidase and Lactoferrins from Bovine Milk Acid Whey by Carboxymethyl Cation Exchange Chromatography. J Dairy Sci. 1991; 74:1439-1444.

1. (canceled)

2. A method of stimulating the immune system of a subject, or increasing an anti-tumour immune response in a subject, or maintaining or improving one or both of the white blood cell count and red blood cell count of a subject, or increasing the responsiveness of a subject to a cancer therapy comprising separate, simultaneous or sequential administration to a subject in need thereof of:
(a) one or more lactoferrin polypeptides, one or more functional lactoferrin variants, one or more functional lactoferrin fragments, one or more metal ion lactoferrin polypeptides, one or more functional metal ion lactoferrin variants, or one or more functional metal ion lactoferrin fragments, or any combination of any two or more thereof, and
(b) one or more anti-tumour food factors selected from vitamin D, one or more vitamin D analogues, soy, soy protein, soy protein concentrate, soy protein isolate, soy milk, soy yoghurt, soy cheese, soy nuts, soybeans, soybean meal, soybean flour, soy butter, or any combination of any two or more thereof.

3. A method of claim 2 that increases the production of Th1 and Th2 cytokines within a tumor of a subject in need thereof.

4. A method of claim 2 that increases the production of Th1 and Th2 cytokines within the intestine of a subject.

5. A method of claim 2 that increases the level of Th1 and Th2 cytokines in the systemic circulation of a subject.

6. (canceled)

7. A method of claim 2 that induces apoptosis of tumour cells in a subject in need thereof.

8. A method of claim 2 that inhibits angiogenesis in a subject in need thereof.

9. A method of claim 2 that inhibits tumour angiogenesis in a subject in need thereof.

10.-11. (canceled)

12. A method of claim 2 that increases the sensitivity of a tumour in a subject to a cancer therapy.

13.-17. (canceled)

18. A method claim 2, further comprising administration of one or more additional anti-tumour food factors selected from vitamin D, one or more vitamin D analogues, soy protein, one or more soybean components, one or more polyphenols, lycopene, wheat bran, one or more flavonoids, inositol, resveratrol, propolis, mushroom extract, anthocyanins, almonds, ginseng, casein hydrolysate, shark cartilage, garlic extracts, selenium supplementation, tea extracts, curcuminoids, caffeine, carnosic acid, capsaicin, sesquiterpene lactones, cotylenin A, humulone, arginine, glutamine, retinoids from green leaf vegetables, cocoa powder, lycopene, glucosinolates from cruciferous vegetables, organosulphur compounds, N-acetyl cysteine, allium compounds, carotenoids, coumarins, dietary fibres, dithiolthiones, indoles, inositol hexaphosphate, isoflavones, isothiocyanates, monoterpenes, diterpene esters, polyphenols, riboflavin 5′ phosphate, cinnamaldehyde, vanillin, umbelliferone, phenols, cinnamic acid, polyphenols, plant sterols, sitostanol, stigmasterol, campesterol), acylglycosylsterols, phytosteroids, protease inhibitors, saponins, isoprenoids, terprenoids, tocotrienols, retinoids, ellagic acid, polyamines, resveratrol, hydroxycinnamic acids (E)-ferulic acid, (E)-p-coumaric acid, chlorophyllin, propolis, one or more components of chlorophyllin, propolis, caffeic acid, phenyl esters, artellipin C, red wine, tannic acid, mushroom extracts, anthocyanins, cyanidins, mushroom beta-glucans, lentinan, spinach leaf extracts, natural antioxidant mixture from spinach leaf, noni juice, vitamins A, vitamin B6, vitamin C, vitamin E, extract of Siamese cassia, extract of Beta vulgaris, extracts of lemon grass, extracts of bamboo grass, carnosic acid, capsaicin, sesquiterpene lactones, parthenolide, costunolide, yomogin, cotylenin A, humulone, omega-3 fatty acids, eicosapentaenoic acid (EPA), docasahexaenoic acid (DHA), or any combination of any two or more thereof.

19.-20. (canceled)

21. A method of claim 2, wherein the metal ion is an ion selected from aluminium, copper, chromium, cobalt, gold, iron, manganese, platinum, ruthenium, zinc ions, or any mixture of any two or more thereof.

22. A method of claim 2, wherein the lactoferrin is from at least about 5% to at least about 10%, metal ion saturated on a stoichiometric basis.

23. A method of claim 2, wherein the lactoferrin is from at least about 25, to at least about 65% metal ion saturated on a stoichiometric basis.

24.-25. (canceled)

26. A method of claim 2, further comprising separate, simultaneous or sequential administration of at least one cancer therapy.

27. A method of claim 26, wherein the cancer therapy is an anti-tumour agent or anti-tumour therapy.

28. A method of claim 26, wherein the anti-tumour therapy is selected from the group consisting, of surgery, chemotherapy, radiation therapy, hormonal therapy, biological therapy, immunotherapy, cellular therapy, anti-angiogenic therapy, cytotoxic therapy, vaccination, nucleic acid-based vaccination, viral-based therapy, gene therapy, small molecule inhibitor therapy, nucleotide-based therapy, antibody-based therapy, oxygen treatment, ozone treatment, embolization, and chemoembolization.

29. A method of claim 2, wherein the lactoferrin and the anti-tumour food factor are administered daily for a period of at least about 1 to at least about 6 weeks before administration of the anti-tumour agent or anti-tumour therapy.

30. A method of claim 2, wherein the lactoferrin and the anti-tumour food factor are administered for period of at least about 1 to at least about 11 before administration of the anti-tumour agent or the anti-tumour therapy.

31. A method of claim 2, wherein the tumour or the cancer is a leukemia, lymphoma, multiple myeloma, a hematopoietic tumor of lymphoid lineage, a hematopoietic tumor of myeloid lineage, a colon carcinoma, a breast cancer, a melanoma, a skin cancer or a lung cancer.

32. A method of claim 30, wherein the tumour is a large tumour.

33. A method of treating or preventing cancer in a subject, the method comprising separate, simultaneous or sequential administration to a subject in need thereof of:
a. one or more lactoferrin polypeptides, one or more functional lactoferrin variants, one or more functional lactoferrin fragments, one or more metal ion lactoferrin polypeptides, one or more functional metal ion lactoferrin variants, or one or more functional metal ion lactoferrin fragments, or any combination of any two or more thereof, and
b. one or more anti-tumour food factors selected from vitamin D, one or more vitamin D analogues, soy, soy protein, soy protein concentrate, soy protein isolate, soy milk, soy yoghurt, soy cheese, soy nuts, soybeans, soybean meal, soybean flour, soy butter, or any combination of any two or more thereof.

34. A composition comprising:
a. one or more lactoferrin polypeptides, one or more functional lactoferrin variants, one or more functional lactoferrin fragments, one or more metal ion lactoferrin polypeptides, one or more functional metal ion lactoferrin variants, or one or more functional metal ion lactoferrin fragments, or any combination of any two or more thereof, and
b. one or more anti-tumour food factors selected from vitamin D, one or more vitamin D analogues, soy, soy protein, soy protein concentrate, soy protein isolate, soy milk, soy yoghurt, soy cheese, soy nuts, soybeans, soybean meal, soybean flour, soy butter, or any combination of any two or more thereof.

35. A method of claim 2, wherein the lactoferrin and the anti-tumour food factor are administered for a period of at least about 1 to at least about 5 weeks before administration of the anti-tumour agent or the anti-tumour therapy.

36. A method of claim 2, wherein the lactoferrin and the anti-tumour food factor are administered for a period of at least about 1 to at least about 4 months before administration of the anti-tumour agent or the anti-tumour therapy.
